(19) United States
(12) Patent Application Publication
(10) Pub. No.: US 2012/0135054 A1
(43) Pub. Date: May 31, 2012

(54) Poly (Ester Ether Amide)s and Uses Thereof

(75) Inventors: Chih-Chang Chu, Ithaca, NY (US); Jun Wu, Ithaca, NY (US); Martha A. Mutschler-Chu, Ithaca, NY (US)

(73) Assignee: CORNELL UNIVERSITY, Ithaca, NY (US)

(21) Appl. No.: 13/381,145

(22) PCT Filed: Jun. 29, 2010
(86) PCT No.: PCT/US10/40403

§ 371 Date: Feb. 15, 2012

Related U.S. Application Data
(60) Provisional application No. 61/221,349, filed on Jun. 29, 2009.

(51) Int. Cl. A61K 47/48; A61K 48/00; A61K 31/7105; C12N 5/02; C08F 126/06; C08F 122/38; C08G 69/44; C08F 114/18; C08F 128/02; A61K 9/14; A61P 35/00; B82Y 5/00

(52) U.S. Cl. 424/400; 424/78.17; 514/44 R; 514/44 A; 435/375; 526/258; 526/304; 528/292; 526/247; 526/288; 528/290; 977/773; 977/788; 977/906

(57) ABSTRACT

Cationic poly(ester ether amide)s (PEEAs) and compositions comprising PEEAs and biomolecules such as nucleic acids and proteins. Also, a method for intracellular delivery of biomolecules using complexes of the PEEAs and biomolecules. For example, PEEAs can be used as transfection agents for nucleic acids such as DNA and RNA.

### CROSS-REFERENCE TO RELATED APPLICATIONS

This application claims priority to U.S. provisional patent application No. 61/221,349, filed Jun. 29, 2009, the disclosure of which is incorporated herein by reference.

### FIELD OF THE INVENTION

The present invention generally relates to preparation and use of amino-acid based polymers and uses thereof. More particularly, the present invention relates to poly(ester ether amides) (PEEAs) and use thereof as delivery agents.

### BACKGROUND OF THE INVENTION

During the past decade, biodegradable, bioresorbable polymers for biomedical uses have garnered growing interest. Recently described, aliphatic poly(ester amide)s (PEAs) based on a-amino acids, aliphatic diols, and fatty dicarboxylic acids have been found to be good candidates for biomedical uses because of their biocompatibility, low toxicity, and biodegradability (K. DeFife et al. Transcatheter Cardiovascular Therapeutics—TCT 2004 Conference. Poster presentation. Washington, D.C. 2004; G. Tsitlanadze, et al. J. Biomater. Sci. Polymer Edn. (2004). 15:1-24).

The highly versatile Active Polycondensation (APC) method, which is mainly carried out in solution at mild temperatures, allows synthesis of regular, linear, polyfunctional PEAs, poly(ester-urethanes) (PEURs) and poly(ester ureas) (PEUs) with high molecular weights. Due to the synthetic versatility of APC, a wide range of material properties can be achieved in these polymers by varying the three components—a-amino-acids, diols and dicarboxylic acids—used as building blocks to fabricate the macromolecular backbone; (R. Katsarava, et al. J. Polym. Sci. Part A: Polym. Chem (1999) 37:391-407).

Delivery of desired biomolecules to cells can be accomplished by various delivery means that generally fall into 4 broad categories: water soluble cationic polymers, lipids, dendrimers and nanoparticles. Among them, the water soluble synthetic and natural polycations have attracted the most attention. A large number of cationic polymers have been tested for gene delivery. Among them, poly-L-lysine (PLL) and polyethylenimine (PEI) have been intensively studied because of their strong interaction with the plasmid DNA which results in formation of a compact polymer/DNA complex. Other synthetic and natural polycations developed as non-viral vectors includes polyamidoamine dendrimers and chitosan, imidazole-containing polymers with proton-sponge effect, membrane-disruptive peptides and polymers like polyethylacrylic acid (PEAA), poly [alpha-(4-aminobutyl)-L-glycolic acid] (PAGA), and poly (amino acid) based materials. However, most of them could not achieve both high transfection efficiency and low toxicity.

Based on the foregoing, there exists and ongoing and unmet need for delivery agents that have high transfection efficiency and low toxicity.

### BRIEF SUMMARY OF THE INVENTION

In one aspect, the present invention provides a polymer having the following structure:

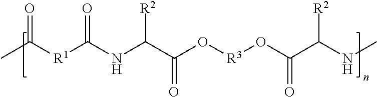

where n is an integer from 2 to 100. R¹ at each occurrence in the polymer is independently selected from C₂ to C₂₀ alkyl or alkenyl. R² at each occurrence in the polymer is independently selected from —(CH₂)_{q}NHC(═NH₂⁺)NH₂ where q at each occurrence in the polymer is from 1 to 5, 4-alkylene imidazolium where the alkylene moiety at each occurrence in the polymer comprises from 1 to 5 carbons and —(CH₂)ₜNH₃⁺ group where t at each occurrence in the polymer is from 1 to 5. In one embodiment, the ammonium ions of the R² groups are present as salts of a weak acid. In one embodiment, the ammonium ions are present as a halide, Tos-, acetate, sulfate, nitrate, or a combination thereof salt. R³ is —(CH₂—(CH₂—O)ₘ—CH₂—CH₂—, and m at each occurrence in the polymer is an integer from 1 to 100. In one embodiment, the polymer has the following structure:

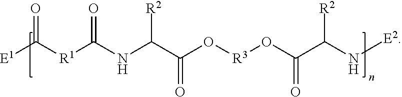

E¹ and E² are independently selected from —H and —OH.

In one aspect, the present invention provides a composition comprising the polymers of the present invention. In one embodiment, the composition further comprises a biomolecule selected from a poly nucleic acid, negatively-charged protein, negatively-charged polysaccharide and a combination thereof. In one embodiment, the poly nucleic acid comprises a gene encoding a peptide or polypeptide. In one embodiment, the poly nucleic acid is RNA. In one embodiment, the RNA is an antisense poly nucleic acid.

In one embodiment, the weight ratio of polymer to poly nucleic acid in the composition is from 50:1 to 12,000:1. In one embodiment, the polymer and poly nucleic acid form a complex and form particles having a size of from 50 nm to 1000 nm. In another embodiment, the particles have a size of from 150 nm to 250 nm.

In one aspect, the present invention provides a method for intracellular delivery of a biomolecule which comprises contacting a cell with the compositions of the present invention herein under conditions suitable to deliver a biomolecule into a cell. In one embodiment, the intracellular delivery of a biomolecule is transfection a poly nucleic acid into a cell. In one embodiment, the compositions comprise a biomolecule selected from a poly nucleic acid, negatively-charged protein, negatively-charged polysaccharide and a combination thereof. In one embodiment, the poly nucleic acid comprises a gene encoding a peptide or polypeptide. In one embodiment, the poly nucleic acid is RNA. In another embodiment, the RNA is an antisense poly nucleic acid. In one embodiment, the cell is a primary cell, stem cell or any other type of cell.

### BRIEF DESCRIPTION OF THE FIGURES

FIG. 1. Example of general synthetic scheme for preparation of PEEA. As an example of reaction conditions: NEt₃ is the catalyst, DMSO is the reaction medium, 75° C. is the reaction temperature and 48 hours is the reaction time.

FIG. 2. Chemical structure of a poly(ester ether amide) (Arg-PEEAs: x-Arg-yEG-z, where x is the number of methylene groups between two closest amide groups and y is the number of ethylene glycol groups between two closest ester groups. z stands for salt type (toluene sulfonic acid salt or chlorine salt)).

FIG. 3. (a) Monomer I: di-p-nitrophenyl ester of dicarboxylic acids; (b) Monomer IIa: tetra-p-toluenesulfonic acid salts of bis-L-Arginine esters; (c) Monomer IIb: dip-toluenesulfonic acid di-hydrochloride acid salts of bis-L-Arginine esters.

FIG. 4. Table 1—List of examples of p-toluenesulfonic acid salt of L-arginine diester from oligoethylene glycols.

FIG. 5. Table 2—Arg-PEEAs (x-Arg-yEG-z) prepared by different combination of diacids and oligoethylene glycol building blocks. NO is made using octanedioic acid, x=6.

FIG. 6. ¹H NMR spectra of 2-Arg-2E-S.

FIG. 7. Table 3-Arg-PEEAs (x-Arg-yEG-z) solubility in distilled water and reduced viscosity in DMSO at room temperature.

FIG. 8. Effect of block length (x) of Arg-PEEAs on the condensation ability to DNA: B means blank, (only 1 μg N3014S DNA, no Arg-PEEA); the other numbers are the WR of Arg-PEEA to DNA. The Arg-PEEAs are: 2-Arg-2E-Cl, 4-Arg-2E-Cl, 8-Arg-2E-Cl (from left to right).

FIG. 9. Effect of block length (y) of Arg-PEEAs on the condensation ability to DNA: B means blank, (only 1 μg N3014S DNA, no Arg-PEEA); the other numbers are the WR of Arg-PEEA to DNA. The Arg-PEEAs are: 2-Arg-2E-Cl, 2-Arg-3E-Cl, 2-Arg-4E-Cl, 2-Arg-6E-Cl and 2-Arg-12E-Cl (from left to right).

FIG. 10. Transfection efficiency of Arg-PEEA/DNA complexes expressed by firefly luciferase activity. The cells used here were primary rat smooth muscle cells (RSMC). Plasmid DNA used were COL (−772)/Luc. Lipofectamine2000® was tested with the optimum WR to DNA. Various WRs of Arg-PEEA to DNA were tested. The number behind the Arg-PEEA name is the WR.

FIG. 11. Table 4—Transfection data.

FIG. 12. GFP Transfection of cells under fluorescence microscope (10×). Green cells are cells successfully transfected with GFP DNA. The cells transfected by lipofectamine2000® were used as controls (12(a)-(c) (left)) and by Arg-PEEA (2-Arg-6E-Cl, WR=1,000) (12(d)-(f) (right)). The cell types from top to bottom are: BAEC, RSMC and MSC; 4 hours treatment for cell lines and 12 hours treatment for primary and stem cells, and images were taken after 48 hours.

FIG. 13. Zeta potential measurements of 2-Arg-6E-Cl/DNA complex in a very wide weight ratio (WR) range. Positive value means the complex is positively charged; while negative value means the complex is negatively charged.

FIG. 14. Particle size measurements of 2-Arg-6E-Cl/DNA complex in a very wide weight ratio (WR) range.

FIG. 15. Cytotoxicity of Arg-PEEA/DNA complexes by MTT assay. Negative control (NC) is cells only without any material treatment. Lipofectamine2000® and PEI were used as positive controls. Four types of Arg-PEEA with various WRs of Arg-PEEA to DNA were tested (2-Arg-2E-S, 2-Arg-6E-S, 2-Arg-2E-Cl, 2-Arg-6E-Cl). The numbers after the PEI and Arg-PEEAs indicate the corresponding WR.

FIG. 16. HUVEC and MSC cell morphology (10×, 12 hours treatment, after 48 hours): (a) Negative control HUVEC, no polymer added; (b) HUVEC cells with 1,000 μg 2-Arg-6E-Cl and 1 μg DNA added; (c) HUVEC cells with 2 μL Liopfectamine2000® and 1 μg DNA added; (c) Negative control MSC, no polymer added; (d) MSC cells with 1,000 μg 2-Arg-6E-Cl and 1 μg DNA added; (e) MSC cells with 2 μL Liopfectamine2000® and 1 μg DNA added.

FIG. 17. Example of PEEA structure—Arg-PEEA-yEG HCL salt (x-A-yE-Cl). yEG: # of ethylene glycol repeating unit in the diol portion of Arg-PEEA. y ranges from 2, 3 and 4 as indicated by 2E, 3E and 4E in FIG. 18. PEG300 and PEG600: Polyethylene glycol of MW 300 and 600 in the diol portion of Arg-PEEAs

FIG. 18. Examples of PEEAs.

FIG. 19. Transfection efficiency of x-A-yE-S firefly luciferase assay (A10 SMC cell line). The RLU/mg value of Superfect is 100.

FIG. 20. Weight ratio of x-A-yE-S to DNA required for maximum transfection efficiency.

FIG. 21. Example of PEEA structure—Arg-PEEA-yEG TsOH salt (x-A-yE-S). yEG: # of ethylene glycol repeating unit in the diol portion of Arg-PEEA. y ranges from 2, 3 and 4 as indicated by 2E, 3E and 4E in FIG. 22. PEG300 and PEG600: Polyethylene glycol of MW 300 and 600 in the diol portion of Arg-PEEAs

FIG. 22. Examples of PEEAs.

FIG. 23. Transfection efficiency of x-A-yE-S firefly luciferase assay (A10 SMC cell line). The RLU/mg value of Superfect is 100.

FIG. 24. Weight ratio of x-A-yE-S to DNA required for maximum transfection efficiency.

FIG. 25. SVEC4-10 endothelial cells cytotoxicity: MTT assay.

### DETAILED DESCRIPTION OF THE INVENTION

The present invention provides polymers comprising cationic poly(ester ether amides) (PEEAs) with polyether moieties, compositions comprising the polymers, and uses of the polymers and/or compositions. In one embodiment, the poly(ester ether amides) are used as delivery (e.g., transfection) agents for delivering biomolecules, including biological macromolecules, (e.g., nucleic acids, proteins and the like) to cells.

The poly(ester ether amides) (PEEAs) of the present invention have, for example, at least the following advantages: (1) diacid and diol parts of the PEEA repeating unit can be selected by use of appropriate monomers; (2) a side chain, such as that of L or D-arginine, which carries a positive charge at physiological pH due to the guanidino group, a very strong basic group with an isoelectric point of 10.96 and pKa about 12.5, which could have a strong potential to condense negatively charged nucleic acids.

In one aspect, the present invention provides cationic PEEAs. The PEEAs are comprised of repeat units, which have at least one amide bond and at least one ester bond, and at least one cationic group, e.g., an ammonium group. The repeat unit also has at least one alkyl polyether moiety. In one embodiment, the polyether group is derived from an oligo(ethylene glycol) group.

In one embodiment, the PEEAs of the present invention have the following structure:

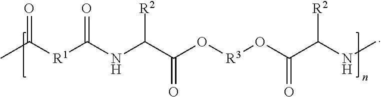

R¹ is an alkyl or alkenyl group and at each occurrence in the polymer it is independently an alkyl or alkenyl group comprising from 2 to 20 carbons, including all integers and ranges therebetween. R¹ is also referred to as a “diacid residue” because, for example, it can be derived from monomer prepared from a diacid. R² is a cationic pendant group and at each occurrence in the polymer it is independently selected from a —(CH₂)_{q}NHC(═NH₂⁺)NH₂ (e.g., the pendant guanidinium group of arginine) where q is from 1 to 5, including all integers therebetween, 4-alkylene imidazolium (e.g., 4-methylene imidazolium) where the alkylene moiety comprises from 1 to 5 carbons, including all integers therebetween, and —(CH₂)ₜNH₃⁺ group where t is from 1 to 5, including all integers therebetween. R² is also referred to as an “amino acid residue” because, for example, it can be derived from a monomer which is prepared from an L or D amino acid. R³ is an alkyl polyether group where each individual alkyl moiety of the polyether comprises from 1 to 8 carbons, including all integers therebetween. R³ is also referred to as a “diol residue” because, for example, it can be derived from a monomer prepared using a diol. For example, R³ can be —(CH₂—CH₂—O)ₘ—CH₂—CH₂— (which can be derived from oligo(ethylene glycol) and m at each occurrence of R³ is an integer from 1 to 100, including all integers and ranges therebetween. In one embodiment, the polymer has the structure shown in FIG. 2. In one embodiment, all R¹ groups in the polymer are the same. In one embodiment, all R² groups in the polymer are the same. In one embodiment, all R³ groups in the polymer are the same. In one embodiment, all the R¹ groups in the polymer are the same, all the R² groups in the polymer are the same and all the R³ groups in the polymer are the same.

The number of repeat units, n, in the polymer is an integer from 2 to 100, including all integers and ranges therebewteen. In one embodiment, the polymer has a molecular weight of from 2000 g/mol to 100,000 g/mol, including all integers and ranges therebetween.

The structure of a 4-alkylene imidazolium (where R⁴ is, for example, —(CH₂)ᵥ— and v is from 1 to 5, including all integers therebetween, or —(CH₂—CH₂—O)_{w} and w is from 1 to 5, including all integers therebetween) is shown below:

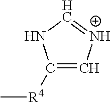

For example, the structure of 4-methylene imidazolium is as follows:

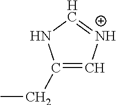

In one embodiment, the PEEAs have one or more counter-ions (e.g., having a pKa from about −7 to +5) associated with positively charged groups therein. Examples of counter-ions suitable to associate with the polymer in the invention composition are counter-anions of weak acids. Examples of such counter-anions include CH₃COO⁻, CF₃COO⁻, CCl₃COO⁻, Tos⁻ (Tos=p-toluene sulfonic acid, ester) and the like. Other examples of suitable counter ions include halides, such as F⁻, Cl⁻ and Br⁻, sulfate and nitrate. In one embodiment, the ammonium groups of the polymer are present as a halide, Tos⁻, acetate, halogen-substituted acetate, sulfate, nitrate, or a combination thereof salt.

In one embodiment, R³ is a moiety which comprises at least one ether functional group. It is a polyether moiety comprising carbon, hydrogen, and oxygen atoms. Accordingly, R³ is not an aliphatic group, such a C₁ to C₆ alkyl group.

In one embodiment, the PEEA has the following structure:

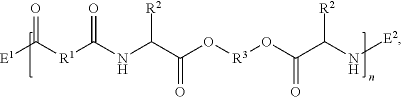

where n, R¹, R², and R³ are as described in the previous embodiment. E¹ and E² are polymer end (or capping) groups. The end groups are functional and can, for example, be used for further chemical modification, such as attachment of copolymer units, small or large biologically active agents like polypeptides, proteins, dyes, nuclear acids, biotin, and the like. Examples of end groups include, but are not limited to, —H, —OH and the like. In one embodiment, the end groups are independently selected from —H and —OH. The choice of end groups can be controlled by adjusting the molar ratio between two monomers (A, diacid monomer, and B, amino acid/diol monomer). For example, if a polymer having one end group as COOH (E¹=OH) and one end group as NH₂ (E²=H) is desired, then the N_{A}/N_{B}(molar ratio) should be equal to 1.00. As another example, if a polymer having two COOH end groups is desired, then the N_{A}/N_{B}(molar ratio) should be greater than 1.00. As yet another example, if a polymer having two NH₂ end groups is desired, then the N_{A}/N_{B}(molar ratio) should be less than 1.00.

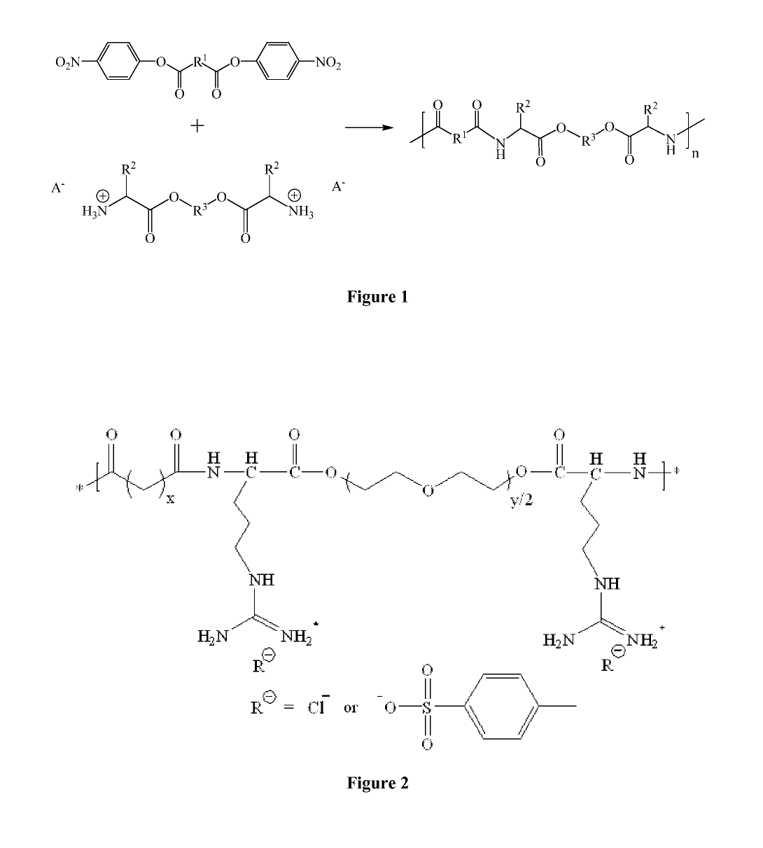

The PEEAs of the present invention can be prepared by polymerization of appropriate monomers. For example, the polymers can be prepared by solution polycondensation of a oligodiol monomer (e.g., a p-toluenesulfonic acid salt of L-arginine oligoethylene glycol diester) and a diester monomer (e.g., di-p-nitrophenyl esters of a dicarboxylic acid). An example of a synthetic scheme for preparation of a PEEA is shown in FIG. 1.

An oligodiol monomer has two amino acids (e.g., two arginine molecules) which are connected via two ester bonds by an alkyl polyether group. Each alkyl moiety of the polyether group has from 2 to 8 carbons, including all integers and ranges therebetween. For example, aryl sulfonic acid salts of diesters of alpha-amino acids and a polyether diol (e.g., oligoethylene glycol) (an oligodiol monomer) can be prepared by admixing alpha-amino acid, e.g., p-aryl sulfonic acid monohydrate, and an polyether diol (e.g., oligoethylene glycol) in toluene, heating to reflux temperature, until water evolution has ceased, then cooling. By “oligoethylene glycol”, as it is used herein, means a low molecular weight polyethylene glycol (e.g., from about 100 g/mol to about 2300 g/mol).

A diester monomer is a diester formed from a saturated dicarboxylic acid. The dicarboxylic acid comprises from 2 to 20 carbons, including all integers and ranges therebetween. The ester groups can be formed from any group which facilitates the polycondensation polymerization reaction of the diester monomer. An example of a suitable ester is a p-nitrophenyl ester. For example, saturated di-p-nitrophenyl esters of dicarboxylic acid and can be prepared as described in U.S. Pat. No. 6,503,538 B1.

In another aspect, the present invention provides a composition comprising a PEEA as described herein. The composition can also comprise a biomolecule. The biomolecule is any molecule that exhibits biological activity and is negatively charged such that it can form a complex with the PEEA. Without intending to be bound by any particular theory, it is considered that the complex is formed by the electrostatic interaction between a positively-charged PEEA and a negatively-charged biomolecule, e.g. a nucleic acid. In various embodiments, the biomolecule is selected from a poly nucleic acid (also referred to as a “polynucleotide”), negatively-charged protein, negatively-charged polysaccharide. In one embodiment, the weight ratio (WR) of polymer to a poly nucleic acid is from 50:1 to 12,000:1, including all integers and ranges between 50 and 12,000.

In one embodiment, the polymer and poly nucleic acid complex are particles having a size of from 50 nm to 1000 nm, including all integers and ranges therebetween. In another embodiment, the polymer and poly nucleic acid complex form particles having a size of from 150 nm to 250 nm, including all integers and ranges therebetween. For example, the size of the particle can be measured as the hydrodynamic radius of the particle in solution. It is desirable that the zeta potential of the particles is from 5 to 15 mV, including all integers and ranges therebetween.

In one embodiment, the biomolecule is a polynucleotide. The polynucleotide used in the compositions, include, for example, deoxyribonucleic acid (DNA), ribonucleic acid (RNA), double or single stranded DNA, double stranded or single stranded RNA, duplex DNA/RNA, and modified polynucleotides.

In one embodiment the poly nucleic acid can be RNA. The term “RNA”, as used herein encompasses any RNA polynucleotide, including but not limited to messenger (mRNA), transfer (tRNA), small interfering (siRNA), short hairpin (shRNA), ribosomal (rRNA), interfering (RNAi), micro RNA and ribozyme.

In another embodiment, the poly nucleic acid can be DNA. “DNA”, as the term is used herein, encompasses any form of DNA. For example, the DNA may be a plasmid or any other type of vector. Non-limiting examples of DNA polynucleotides that can be used in the invention include shuttle vectors, cloning vectors, expression vectors, cosmids, bacterial artificial chromosomes (BACs), yeast artificial chromosomes (YACs) and the like.

In one embodiment, the poly nucleic acid is an expression vector. The PEEA complex expression vectors can be used in a method of the invention to transfect cells, wherein subsequent to transfection, a gene present in the expression vector is expressed in the cell into which the vector was transfected. The expressed gene can be any gene, such as a reporter gene. Examples of reporter genes include but are not limited to luciferase, green fluorescent protein (GFP) and enhanced GFP (EGFP). Expression of the gene can also provide a prophylactic and/or therapeutic benefit to the cell, tissue, organ or individual in which the gene is expressed.

In one embodiment, the invention provides for delivery of a polynucleotide (such as RNA) into one or more cells, whereby the polynucleotide can participate in post-transcription gene silencing (PTGS) of one or more target genes. Thus, in various embodiments, the invention provides compositions and methods for use in interfering RNA (RNAi)-mediated PTGS.

The poly nucleic acid used in the invention can have any suitable length. Specifically, the poly nucleic acid can be about 2 to about 5,000 nucleotides in length, including all integers from 2 to 5,000; about 2 to about 1,000 nucleotides in length, including all integers from 2 to 1,000; about 2 to about 100 nucleotides in length, including all integers from 2 to 100; or about 2 to about 10 nucleotides in length, including all integers from 2 to 10. An antisense poly nucleic acid is typically a poly nucleic acid that is complimentary to an mRNA that encodes a target protein.

A polynucleotide used in the invention can also be a “poly nucleic acid decoy.” The term “poly nucleic acid decoy”, as used herein refers to a poly nucleic acid that inhibits the activity of a cellular factor upon binding of the cellular factor to the poly nucleic acid decoy. The poly nucleic acid decoy contains the binding site for the cellular factor. Examples of such cellular factors include, but are not limited to, transcription factors, polymerases and ribosomes. An example of a poly nucleic acid decoy for use as a transcription factor decoy will be a double-stranded poly nucleic acid containing the binding site for the transcription factor. Alternatively, the poly nucleic acid decoy for a transcription factor can be a single-stranded nucleic acid that hybridizes to itself to form a snap-back duplex containing the binding site for the target transcription factor. An example of a transcription factor decoy is the E2F decoy. E2F plays a role in transcription of genes that are involved with cell-cycle regulation and that cause cells to proliferate. Controlling E2F allows regulation of cellular proliferation. For example, after injury (e.g., angioplasty, surgery, stenting) smooth muscle cells proliferate in response to the injury. Proliferation may cause restenosis of the treated area (closure of an artery through cellular proliferation). Therefore, modulation of E2F activity allows control of cell proliferation and can be used to decrease proliferation and avoid closure of an artery. Examples of other such poly nucleic acid decoys and target proteins include, but are not limited to, promoter sequences for inhibiting polymerases and ribosome binding sequences for inhibiting ribosomes. It is understood that the invention includes poly nucleic acid decoys constructed to inhibit any target cellular factor.

Poly nucleotide used in the compositions/methods of the invention can be synthesized according to commonly known chemical methods or can be obtained from a commercial supplier. The poly nucleic acid can include at least one nucleotide analog, such as bromo derivatives, azido derivatives, fluorescent derivatives and combinations thereof. Nucleotide analogs are well known to those of skill in the art. The poly nucleic acid can include a chain terminator. The poly nucleic acid can also be used, e.g., as a cross-linking reagent or a fluorescent tag. Many common conjugations can be employed to couple a poly nucleic acid to another moiety, e.g., phosphate, hydroxyl, etc. Additionally, a moiety may be linked to the poly nucleic acid through a nucleotide analog incorporated into the poly nucleic acid. In another embodiment, the poly nucleic acid can include a phosphodiester linked 3′-5′ and 5′-3′ poly nucleic acid backbone. Alternatively, the poly nucleic acid can include non-phosphodiester conjugations, such as phosphothioate type, phosphoramidate and peptide-nucleotide backbones. In another embodiment, moieties can be linked to the backbone sugars of the poly nucleic acid. Methods of creating such conjugations are well known to those of skill in the art.

For example, the condensed polymer/poly nucleic acid (i.e., polymer/poly nucleic acid complex) can degrade in vitro in the presence of an enzyme, such as arginase or esterase, or when injected in vivo to provide time release of a suitable and effective amount of the poly nucleic acid. Typically, the suitable and effective amount of poly nucleic acid can be released in a time range from about twenty-four hours to about seven days. Any suitable and effective period of time can be chosen by judicious selection of certain factors. Factors that typically affect the length of time over which the poly nucleic acid is released from the invention composition include, e.g., the nature and amount of polymer, the nature, size and amount of poly nucleic acid, the pH, and the temperature and electrolyte or enzyme content of the environment into which the composition is introduced.

It is desirable the complex be soluble in biologically relevant media. For example, it is desirable that the complex be soluble in water and other aqueous conditions, such as blood, serum, tissue, and the like, and in water/alcohol mixtures. As used herein, the terms “water solubility” and “water soluble” as applied to the invention gene transfer compositions means the concentration of the composition per milliliter of deionized water at the saturation point of the composition therein. Water solubility will be different for each different polymer, but is determined by the balance of intermolecular forces between the solvent and solute and the entropy change that accompanies the solvation. Factors such as pH, temperature and pressure will alter this balance, thus changing the solubility. The solubility is also pH, temperature, and pressure dependent.

As generally defined, water soluble polymers can include truly soluble polymers to hydrogels (G. Swift, Polymer Degr. Stab. 59: (1998) 19-24). Invention compositions can be scarcely soluble (e.g., from about 0.01 mg/mL), or can be hygroscopic and when exposed to a humid atmosphere can take up water quickly to finally form a viscous solution in which composition/water ratio in solution can be varied infinitely.

The solubility of the polymers used in invention gene transfer compositions in deionized water at atmospheric pressure is in the range from about 0.01 mg/mL to 400 mg/mL at a temperature in the range from about 18° C. to about 55° C., preferably from about 22° C. to about 40° C. Quantitative solubility of the invention compositions can be visually estimated according to the method of Braun (D. Braun et al. in Praktikum der Makromolekularen Organischen Chemie, Alfred Huthig, Heidelberg, Germany, 1966). As is known to those of skill in the art, the FloryHuggins solution theory is a theoretical model describing the solubility of polymers. The Hansen Solubility Parameters and the Hildebrand solubility parameters are empirical methods for the prediction of solubility. It is also possible to predict solubility from other physical constants, such as the enthalpy of fusion.

The water solubility of the invention compositions can also be characterized using such assays as static light scattering and size exclusion chromatography (SEC). Additionally, polymers can be characterized by ¹H NMR, ¹³C NMR, gel permeation chromatography (GPC), and differential scanning calorimetry (DSC), as is known in the art and as illustrated in the Examples herein.

In one embodiment, the PEEA—biomolecule complex can be prepared by condensation of a PEEA with a biomolecule (e.g., a poly nucleic acid). For example, the desired weight ratio of PEEA and biomolecule are admixed in a solution (e.g., an appropriate buffer solution). The solution is thoroughly mixed (e.g., by vortex mixing for 5 to 30 seconds) at room temperature. The solution is then allowed to equilibrate to ambient conditions (e.g. by allowing the mixture to stand for 20 to 30 minutes.

In one aspect, the invention provides a method for introducing a biomolecule into a cell. In one embodiment, the present invention provides a method for intracellular delivery of a biomolecule. In one embodiment, the method comprises the step of contacting a cell with a PEEA-biomolecule complex as described herein under conditions suitable to deliver a biomolecule into a cell. Concurrent with or subsequent to contacting the cell, the biomolecule enters the cell. The method may be performed to introduce the biomolecule to a cell in vitro or in vivo.

In general, contacting the cell with a composition of the invention comprises either administering a composition of the invention to an individual, wherein the composition comes into contact with cells in the individual and the biomolecule enters the cell, or contacting cells in vitro with a composition of the invention. In the former case, the composition may be administered to the individual using any available method and route, including oral, parenteral, subcutaneous, intraperitoneal, intratumoral, intrapulmonary, intranasal and intracranial injections. Parenteral infusions include intramuscular, intravenous, intraarterial, intraperitoneal, and subcutaneous administration.

In one embodiment, a composition of the invention can be injected directly into a tissue that comprises a target gene, the inhibition of expression of which is desirable.

Administration of the compositions can be performed in conjunction with conventional therapies that are intended to treat a disease or disorder for which the biomolecule is expected to provide a therapeutic and/or prophylactic benefit. Thus, in various embodiments, the individual to whom a composition of the invention is administered can be an individual that is in need of treatment for any disease or disorder. In one embodiment, the individual is in need of therapy for a cancer. In non-limiting embodiments, the cancer can be a solid tumor or a blood cancer.

For introducing a biomolecule into a cell in vitro, the method generally includes incubating the cells with a composition of the invention for a period of time sufficient for the biomolecule to enter the cell. The method may further comprise incubating cells in a suitable cell culture media and/or a suitable buffer, either of which can be performed before, after or concurrent with incubation of the cells with the composition of the invention. Those skilled in the art, given the benefit of the present invention, will be able to determine suitable buffers and incubation times, and amounts/concentrations of the molecule, depending on the type and amount of molecule being introduced to the cells, the type and density of the cells, the method of introduction, and other conditions that will be apparent to the skilled artisan.

It will accordingly be recognized by those skilled in the art that virtually any cell type, and particularly prokaryotes, fungi, insects, mammalian and avian cells, are suitable for receiving biomolecules delivered using the compositions and the methods of the invention. For example, the cell can be: cells from cell lines such as SMC A10, NRK49, Human Detroit 539 fibroblast cells, SVEC4-10, BAEC endothelial cells and RAW 264.7 macrophages; primary cells (such as rat, mouse and human smooth muscle cells, rat aortic fibroblast cells (RAF), human umbilical vein endothelial cells (HUVEC)); and stem cells (such as Mesenchymal stem cells (MSC) and rat bone marrow cells (BM)).

In one embodiment, the PEEA-biomolecule complex is a PEEA—poly nucleic acid complex and the method results in transfection of the poly nucleic acid. In one embodiment, a primary or stem cell is transfected with a poly nucleic acid. No detectable transfection is observed for primary or stem cells when PEAs with an aliphatic diol residue are used. Without intending to be bound by any particular theory, it is considered that the improved transfection efficiency results from use of a PEEA-biomolecule complex with specific particle size and zeta potential. The transfection efficiency can be determined by physico-chemical tests (such as gel electrophoresis, fluorescence, and luciferase expression assays).

The PEEAs of the present invention exhibit desirable intracellular delivery of biomolecules with lower levels of cytotoxicity compared to currently used delivery agents (such as Superfect® and Lipofectamine 2000®) as further described in the example below.

The following examples are presented to illustrate the present invention. They are not intended to limiting in any manner.

### Example 1

### Synthesis, Characterization, and Use of Poly(ester amide)s as Transfection Agents

A series of Arg-PEEAs (FIG. 2) having different methylene (x)/ethylene glycol (y) chain length (x=2, 4, 8; y=2, 3, 4, 6, 12) in the repeating unit were prepared.

### Materials

L-Arginine, L-Arginine hydrochloride, p-toluenesulfonic acid monohydrate, succinyl chloride, adipoyl chloride, sebacoyl chloride, di-ethylene glycol (DEG), tri-ethylene glycol (TEG), tetra-ethylene glycol (TTEG), poly (ethylene glycol) (Mn=300), poly (ethylene glycol) (Mn=600), triethylamine and p-nitrophenol were all purchased from Alfa Aesar (Ward Hill, Mass.) and used without further purification. Organic solvents like methanol, toluene, ethyl acetate, acetone, 2-propanol and dimethyl sulfoxide (DMSO) were purchased from VWR Scientific (West Chester, Pa.) and were purified by standard methods before use. Other chemicals and reagents if not otherwise specified were purchased from Sigma (St. Louis, Mo.).

Polyethylenimine (PEI) with a reported weight average molecular weight (M_{w}) of 25,000, ethidium bromide, MTT, Dulbecco's phosphate-buffered saline (PBS, pH 7.4), TAE, HEPES and other buffers were purchased from Sigma (St. Louis, Mo.). Dulbecco's modified eagle medium (DMEM), penicillin—streptomycin (PS, 100 U/mL), trypsin—EDTA (TE, 0.5% trypsin, 5.3 mM EDTA tetra-sodium), fetal bovine serum (FBS) were obtained from Gibco BRL (Rockville, Md.). Cell lines (SMC A10, NRK49, Human Detroit 539 fibroblast cells, SVEC4-10, BAEC endothelial cells, RAW 264.7 macrophages), primary cells (Rat, Mouse and Human smooth muscle cells, Rat aortic fibroblast cells (RAF), Human umbilical vein endothelial cells (HUVEC)) and stem cells (Mesenchymal stem cells (MSC) and rat bone marrow cells (BM)) were obtained from American Type Culture Collection (ATCC, Manassas, Va.) or Professor Bo Liu's lab at Surgery Department of Wisconsin University. DNA size marker N3014 was purchased from New England Lab (Woburn, Mass.). A Qiagen endotoxin-free plasmid Maxi kit was purchased from Qiagen (Valencia, Calif.). Lipofectamine2000® was purchased from Invitrogen (Carlsbad, Calif.). Promega Luciferase Assay Kit containing luciferase cell culture lyses reagent and luciferase substrates were obtained from Promega (Madison, Wis.).

### Synthesis of Monomers and Polymers

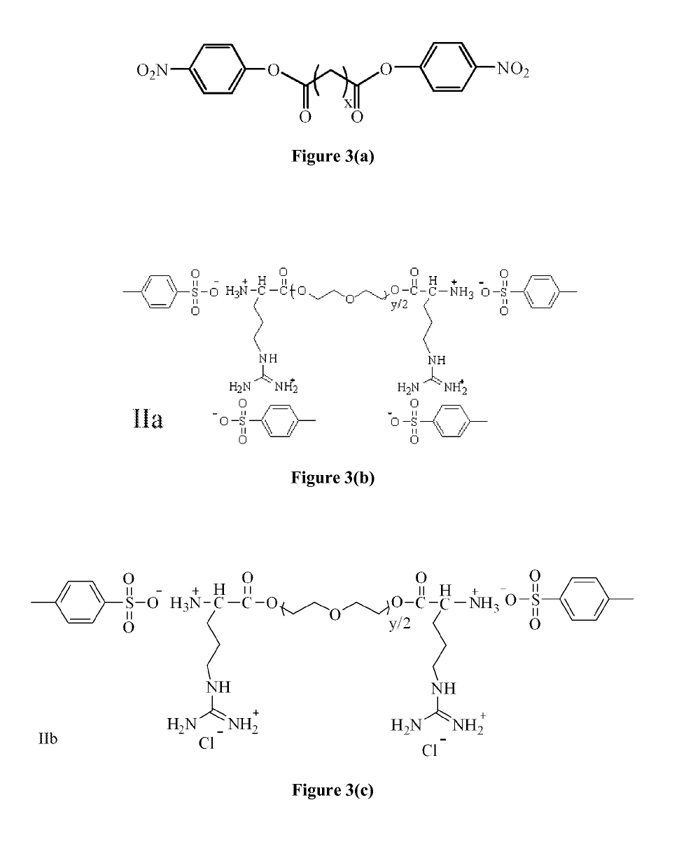

The general scheme of Arg-PEEA synthesis was divided into the following three major steps: the preparation of di-p-nitrophenyl ester of dicarboxylic acids (I) (FIG. 3a); the synthesis of p-toluenesulfonic acid salt of L-arginine diester (II) from di-ethylene glycol, tri-ethylene glycol, and tetra-ethylene glycol, PEG300 and PEG600 (the preparation of tetra-p-toluenesulfonic acid salts of bis-L-Arginine esters (IIa) (FIG. 3b) and the preparation of di-p-toluenesulfonic acid di-hydrochloride acid salts of bis-L-Arginine esters (IIb)) (FIG. 3c); and the synthesis of Arg-PEEAs (III) (FIG. 2) via the solution polycondensation of monomers (I) and (II).

Di-p-nitrophenyl esters of dicarboxylic acids (Monomer I) were prepared by reacting dicarboxylic acyl chloride varying in methylene length (x) with p-nitrophenol. Three monomers were prepared: di-p-Nitrophenyl Succinate (NSu with x=2); di-p-Nitrophenyl Adipate (NA with x=4); di-p-Nitrophenyl Sebacate (NS with x=8). x indicates the numbers of methylene group in the diacid.

L-arginine is used for the preparation of tetra-p-toluenesulfonic acid salts of bis-L-Arginine esters (IIa). Because of the strong positive charge characteristic of L-arginine, the amount of p-toluenesulfonic acid used for the synthesis of p-toluenesulfonic acid salt of L-arginine diester was doubled when compared with the prior synthesis of p-toluenesulfonic acid salt of non-ionic hydrophobic amino acids diesters. The need to double the amount of p-toluenesulfonic acid in the current case is because of the preferential consumption of the p-toluenesulfonic acid by the strong basic guanidine group on L-arginine side chain.

For example, L-arginine (0.04 mol) and di-ethylene glycol (0.02 mol) were directly mixed in a three neck round bottom flask with toluene (b.p. 110° C.) (400 mL) with the presence of p-toluenesulfonic acid monohydrate (0.082 mol). The solid-liquid reaction mixture was heated to 130° C. and reflux for 24 hr after 2.16 mL (0.12 mol) of water was generated. The reaction mixture (viscous solid) was then cooled to room temperature. Toluene was decanted. The dried reacted mixture was finally purified by repeated precipitation in 2-propanol for three times. 2-propanol was decanted afterwards, and then the white sticky mass was dried in vacuum. Five monomers (IIa) were made (Table 1): Arg-2E-S, Arg-3E-S, Arg-4E-S, Arg-6E-S, and Arg-12E-S. 2E, 3E, 4E stand for the di-ethylene glycol, tri-ethylene glycol and tetra-ethylene glycol, respectively; 6E stands for PEG300 because the number of ethylene glycols of PEG300 is around 6-7; 12E stands for PEG600 because the number of ethylene glycols of PEG600 is around 12-13. The first 3 monomers (Arg-2E-S, Arg-3E-S, and Arg-4E-S) are white solid powder and the last two are transparent or yellow viscous solid. All of them are obtained in high yields (80˜90%).

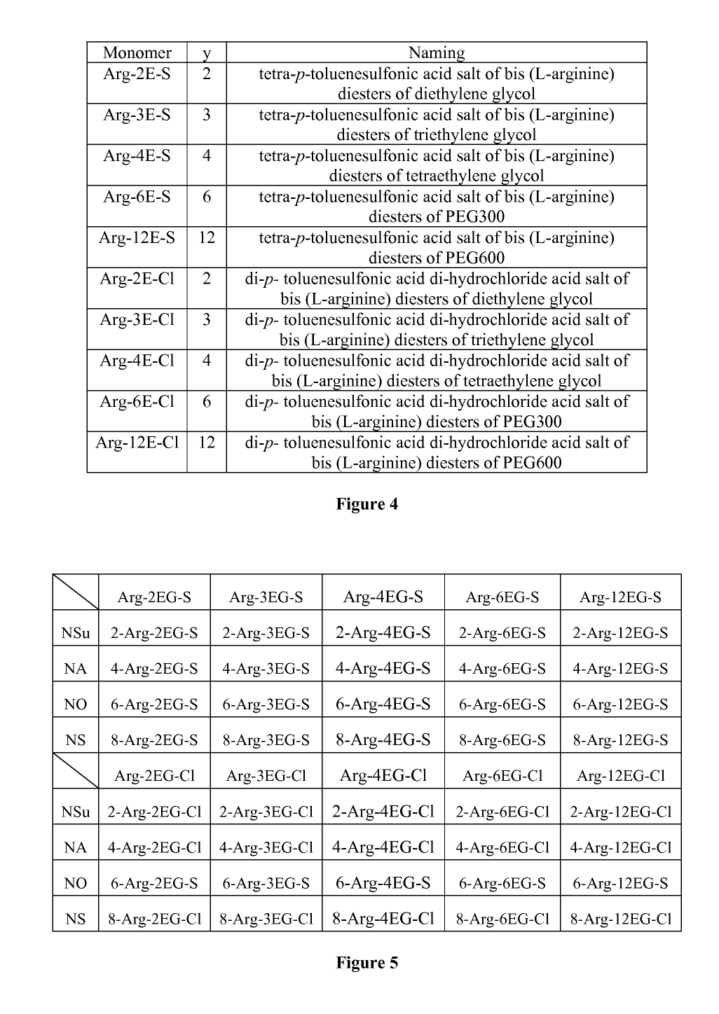

L-Arginine hydrochloride is used for the preparation of di-p-toluenesulfonic acid di-hydrochloride acid salts of bis-L-arginine esters (IIb). Since the basic guanidine group on arginine side chain has formed the salt with hydrochloride acid, the amount of p-toluenesulfonic acid used for the synthesis of di-p-toluenesulfonic acid di-hydrochloride acid salts of bis-L-arginine esters was same as the prior synthesis of p-toluenesulfonic acid salt of non-ionic hydrophobic amino acids diesters. Five monomers (IIb) were made in this study (Table 1): Arg-2E-Cl, Arg-3E-Cl, Arg-4E-Cl, Arg-6E-Cl, and Arg-12E-Cl. The definitions of 2E, 3E, 4E, 6E and 12E are same as the above paragraph. The first 3 monomers are white solid powder and the last two are transparent or yellow viscous solid. All of them are obtained in high yields (80˜90%). All the prepared monomers (IIa and IIb) are listed in Table 1 (FIG. 4) and labeled as Arg-yEG-z, where y are the number of ethylene glycol units in diols and z is the salt type (S (toluene sulfonic acid salt) or Cl (hydrochloride salt)).

Arg-PEEAs (FIG. 2) were prepared by a solution polycondensation of the above (I) and (II) monomers at different combinations in DMSO solvent and the prepared Arg-PEEAs are listed in Table 2 (FIG. 5). All the Arg-PEEAs are labeled as x-Arg-yEG-z, where x and y are the number of methylene groups in diacids and ethylene glycol units in diols, respectively, and z is the salt type (S (toluenesulfonic acid salt) or Cl (hydrochloride salt)). An example of the synthesis of 2-Arg-2E-S via solution polycondensation is given here. Monomers NSu (1.0 mmol) and Arg-2E-S (1.0 mmol) in 1.5 mL of dry DMSO were mixed well by vortexing. The mixture solution was heated up with stirring to obtain a uniform mixture. Triethylamine (0.31 mL, 2.2 mmol) was added drop by drop to the mixture while heating up to 75° C. with vigorous stirring until the complete dissolution of the monomers. The solution color turned into yellow after several minutes. The reaction vial was then kept for 48 hrs at 75° C. in a thermostat oven without stirring. The resulting solution was precipitated in cold ethyl acetate, decanted, dried, re-dissolved in methanol and re-precipitate in cold ethyl acetate for further purification. Repeat the purification for 2 times before drying in vacuo at room temperature. The prepared Arg-PEEAs are white solid powder (for EG with number 2, 3 and 4) or transparent/yellow viscous solid (for EG with number 6 and 12). All of them are obtained in high yields (80˜90%).

### Measurement Methods

The physicochemical properties of the prepared monomer and polymers were characterized by various standard methods. For Fourier transform infrared (FTIR) characterization, the samples were ground into powders and mixed with KBr at a sample/KBr ratio of 1:10 (w/w). FTIR spectra were then obtained with a PerkinElmer (Madison, Wis.) Nicolet Magana 560 FTIR spectrometer with Omnic software for data acquisition and analysis. ¹H NMR spectra were recorded with a Varian Unity Inova 400-MHz spectrometer (Palo Alto, Calif.). Deuterated water (D₂O-d₂; Cambridge Isotope Laboratories, Andover, Mass.) with tetramethylsilane as an internal standard or deuterated dimethyl sulfoxide (DMSO-d₆; Cambridge Isotope Laboratories) was used as the solvent. MestReNova software was used for the data analysis. The thermal properties of the synthesized Arg-PEEAs were characterized with a DSC 2920 (TA Instruments, New Castle, Del.). The measurements were carried out from −20 to 200° C. at a scanning rate of 10° C./min and at a nitrogen gas flow rate of 25 mL/min. TA Universal Analysis software was used for thermal data analysis. The solubility of Arg-PEEAs in common organic solvents at room temperature was assessed by using 2.0 mg/mL as a solubility standard to determine whether Arg-PEEA polymer is soluble or not in a solvent. The quantitative solubility of Arg-PEEAs in distilled water at room temperature was measured by adding distilled water step by step until the clear solution was obtained. The reduced viscosity (η_{red}) of the polymers synthesized was determined by a Cannon-Ubbelhode viscometer in DMSO solution at a concentration of 0.25 g/dL at 25° C.

Electrophoresis Assay. The Arg-PEEA/DNA complexes for agarose gel electrophoresis assay were prepared by adding the DNA marker (N3014 DNA maker) solution into the Arg-PEEA aqueous solutions (in 1×PBS buffer). After mixing the two solutions together, it was immediately vortex for 2-3 seconds, and then equilibrated at an ambient condition for 30 minutes. Arg-PEEA/DNA complexes were analyzed by electrophoresis in a 1 agarose gel stained with ethidium bromide (10 μg/mL) with TAE buffer at 100 V for 90 min. Total injection volume was 15 μL which consisted of 2 μL blue dye solution, 2 μL DNA marker solution (500 μg/mL), several μL of the Arg-PEEA polymer PBS solution and several μL of pure PBS buffer solution. The Arg-PEEA solutions must be made freshly or stored at 4° C. before use. The amount of DNA was fixed at 1 μg per test. After mixing all the solutions, the final system was shaken or centrifuged heavily for several seconds. The N3014 DNA maker solution without Arg-PEEA was used as a blank control. The N3014 DNA marker was visualized by an UV illumination (FOTO/UV 300 Transilluminator). The migration of DNA from the Arg-PEEA/DNA complex was recorded by a digital camera (Panasonic WV-BP330).

Cell Culture. In this report, the following cells were used for tests: cell lines (SMC A10, NRK49, Human Detroit 539 fibroblast cells, SVEC4-10 and BAEC endothelial cells, RAW 264.7 macrophages), primary cells (Rat, Mouse and Human smooth muscle cells, Rat aortic fibroblast cells (RAF), Human umbilical vein endothelial cells (HUVEC)) and stem cells (Mesenchymal stem cells (MSC) and rat bone marrow cells (BM)). All the cells were grown exactly as the recommended ATCC protocols. For examples, the rat SMC A10 cell lines was grown as recommended at 37° C. in 5% CO₂ in Dulbecco's minimal essential medium (DMEM) supplemented with 10% FBS and antibiotics. The cell lines were used from passages 6 to 12 and primary cells and stem cells were used from passages 2-5. Media was changed every 2 days. Cells were grown to 70% confluence before splitting, harvesting or transfection.

### Preparation of Plasmid DNA and Complexes of Arg-PEEA/DNA

The luciferase encoding reporter plasmids, COL (−772)/LUC and green fluorescence protein encoding reporter plasmid DNA (GFP) were all provided by Dr. Bo Liu's lab at Surgery Department of Wisconsin University at Madison. All plasmids were prepared using Qiagen endotoxin-free plasmid Maxi kits according to the supplier's protocol. The quantity and quality of the purified plasmid DNA was assessed by spectrophotometric analysis at 260 and 280 nm as well as by electrophoresis in 1% agarose gel. Purified plasmid DNA were resuspended in TAE (Tris-acetate-EDTA) buffer and frozen in −20° C. The DNA solution obtained had a concentration around 1.5-2.0 mg/mL and was diluted to around 0.5 mg/mL before use.

The Arg-PEEA/DNA complexes were prepared by adding the plasmid DNA buffer solution into the freshly prepared Arg-PEEA PBS buffer solutions at a room temperature to obtain a desirable Arg-PEEA to DNA weight ratio (WR). In this report, a wide range of WR (from 50 to 3,000) of Arg-PEEA to DNA was tested. The mixed solution was immediately and slightly vortex for several seconds and then equilibrated at an ambient condition for 20-30 minutes. All the Arg-PEEA solutions and Arg-PEEA/DNA complexes were freshly prepared and used within 4 hours.

### Zeta Potential and Particle Size Measurements for Arg-PEEA/DNA Complexes

The charge properties of the Arg-PEEA/DNA complexes were studied by zeta potential measurements. Arg-PEEA solutions (2 mg/mL) were prepared by dissolving Arg-PEEAs in 1×PBS buffer solution and the solution was filtered (0.45 μm pore size, Whatman®) before experiments. The Arg-PEEA/DNA complexes were prepared by adding the plasmid DNA (N3012, New England Lab) buffer solution of pre-determined amounts to the freshly made Arg-PEEA PBS buffer solutions (1 mL volume total) to obtain desirable Arg-PEEA to DNA weight ratio (WR). The mixed solution was immediately and slightly vortex for several seconds, and then equilibrated at an ambient condition for 20 minutes. After that, the zeta potential of the Arg-PEEA/DNA complexes was measured at 25° C. by using a Malvern Zetasizer Nano-ZS machine (Worchestershire, UK). Zeta potentials were calculated by using the Smoluchowsky model for aqueous suspensions. 2-Arg-6E-Cl was selected for this study and 2-Arg-6E-Cl/DNA complexes with a series of WR were measured.

Meanwhile, the particle sizes of the Arg-PEEA/DNA complex were studied by the same Malvern Instruments Zetasizer Nano ZS instrument, which used light scattering to measure the average hydrodynamic radius of particles in solution. Samples were placed in 1.0 mL plastic cuvettes and three measurements consisting of 50 runs with 5 s duration were performed at 25° C. The instrument was standardized with 1 mm polystyrene beads and particle size was reported as the average of the three measurements with an error measurement of one standard deviation. 2-Arg-6E-Cl was selected for this study and 2-Arg-6E-Cl/DNA complexes with a series of WR were measured.

### Gene Transfection and Luciferase Assay

The complexes formed between plasmid DNA and the Arg-PEEAs were assessed for their in vitro transfection activity utilizing a transient expression of luciferase reporter in cells. First, the transfection protocol for Arg-PEEAs was studied and optimized in terms of cell type, cell density, buffer types, transfection time, transfection media, and temperature. After optimization, all transfection experiments were carried out according to the optimized protocol.

The details for the optimized transfection protocol for Arg-PEEAs are given below. For cell lines, such as SMC A10 cells, the cells were seeded in 0.5 mL complete DMEM (10% FBS, 1% Hepes, 1% penicillin-streptomycin) at 30×10³ per well in a 24-well plate 24 hours before transfection (70% confluent at transfection). Before transfection, the cell culture media was removed and the cells were washed with PBS buffer twice. Then 1.0 mL warmed serum free DMEM media (without antibiotics) was added into each well. For Lipofectamine2000, the media was used according to the manufacturer's recommendation. The formulated Arg-PEEA/DNA complex solution was then added into each well. The plasmid DNA amount was fixed at 1 μg per well for 24-well cell culture plate. The transfection mixtures were immediately and slightly piped up and down for a few seconds, the cells were transfected for 4 hours at 37° C. (5% CO₂) in an incubator, and then the media solution was removed. After that, 0.5 mL of complete DMEM (10% FBS, 1% Hepes, 1% penicillin-streptomycin) were added into each well and kept incubated at 37° C. (5% CO₂) in an incubator. After 48 hours, cells were harvested for luciferase reading. Triplicate results were obtained in each data point. The main differences between transfection of cell lines and primary cells/stem cells were the transfection time and cell culture media: for transfection time: 4 hours is needed for cell lines and 12-16 hours is needed for primary and stem cells; for cell culture media, the transfection media is the media recommended by ATCC without serum, the medias before and after transfection are the medias recommended by ATCC.

Gene expression was then determined by the luciferase activity using a DT 20/20 luminometer (Turner Biosystems, Sunnyvale, Calif.) with Dual Luciferase Assay System (Promega) according to the manufacturer's instruction. Luciferase assay was performed according to Promega's recommendation. Briefly, cells from each well of a 24-well plate were lysed in 100 μL lysis buffer, transferred to a micro-tube, and then centrifuged at 10,000 g for 2 min. Supernatants were collected and analyzed for luciferase activity. In a typical experiment, 20 μL of supernatant was added to luminometric tubes containing 100 μL of luciferase substrate (Promega). Light emission was measured with a Dual-luciferase detection system for periods of 5 seconds, and the relative light units (RLUs) were determined. Triplicate results were used in each experiment. RLUs were normalized to the protein contents of each sample measured by spectrophotometric analysis.

### Green Fluorescence Protein (GFP) Assay

To visually confirm the transfection efficiency obtained from the luciferase activity reading, we also transfected the many types of cells with a plasmid DNA encodes for Green Fluorescent Protein (GFP). The transfection protocol was exactly the same as the one used for luciferase assay, except GFP encoded plasmid DNA was used. After 48 hours incubation after transfection, cells were examined under a fluorescence microscope (Nikon TE2000-U DIC inverted microscope with UV, GFP/FITC and Tx Red filter sets) for any GFP expression (cells showed green). The cell images were recorded from the random but typical fields of the cell culture wells.

### Evaluation of Cytotoxicity of the Arg-PEEA/DNA Complexes

The evaluation of the cytotoxicity of Arg-PEEA/DNA complexes was performed by MTT assay. All the cell types were tested for this study. The following are the details of cytotoxicity test: The cultured cells were seeded at an appropriate cell density concentration (3,000 or 5,000 cells/well) in 96-well plates and incubated overnight in a 5% CO₂ incubator at 37° C. The cells were, then, treated with various Arg-PEEA/DNA complex solutions for 4 hours or 12 hours. The media was removed after that and complete DMEM was then added. Cells treated only with normal cell culture media were used as the negative control (NC). PEI and Lipofectamine2000® treated cells (same time as the Arg-PEEA/DNA complexes) were used as the positive controls. After 48 hours incubation at 37° C. and 5% CO₂, 15 μL of MTT solution (5 mg/mL) was added to each well, followed by 4 hours incubation at 37° C., 5% CO₂. The cell culture medium including complex solution was carefully removed and 150 μL of acidic isopropyl alcohol (with 0.1 M HCl) was added to dissolve the formed formazan crystal. OD was measured at 570 nm (subtract background reading at 690 nm) using a VersaMax Tunable Microplate reader. The cell viability (%) was calculated according to the following equation: Viability (%)=(OD_{570 (sample)}−OD_{620 (sample)}/(OD_{570 (control)}−OD_{620 (control)}×100%; where the OD_{570 (control)} represented the measurement from the wells treated with medium only, and the OD_{570 (sample)} from the wells treated with various Arg-PEEA and Arg-PEEA/plasmid DNA complexes. Thus, the cell viability was expressed as the percentage of the blank negative control. Triplicates were used in each experiment.

### Statistics

Where appropriate, the data are presented as SEM (mean±standard error) of the mean calculated over at least three data points. Significant differences compared to control groups were evaluated by unpaired Student's t-test or Dunnet test at p 0.05, and between more than two groups by Tukey's test with or without one-way ANOVA analysis of variance. JMP software (version 8.0, from SAS Company) was used for data analysis.

### Results and Discussion

The goal was to examine a new generation of Arg-PEEA, oligoethylene glycol based Arg-PEA (Arg-PEEA), for gene delivery applications, especially for the transfection of primary cells and stem cells, which are hard to be transfected with high efficiency and low cytotoxicity simultaneously. And we also want to examine the relationship of polymer structure-function. We focused on a few chemical structure parameters of Arg-PEEAs (i.e., x (the number of CH₂ groups in the diacid part) and y (the number of ethylene glycol (—CH₂CH₂O—) groups in the diol part) and how the introduction of ethylene glycol groups could affect Arg-PEEAs' properties and their transfection performance, compared with aliphatic diol based Arg-PEAs. Our current PEG approach is different from others' published studies of PEG involved gene transfection which have focused on the modification of the side chain of existing polymers or making amphiphilic block copolymers to increase the transfection efficiency or cell viability. Based on our results, a very stiff backbone (with double bonds in repeating unit, not the side group) in the Arg-PEA main chain would cause significant decrease of transfection efficiency of Arg-PEAs. Here we show that a soft backbone (with ethylene glycol in repeating unit, not the side group) has desirable transfection performance.

### Synthesis and Physicochemical Characterization of Arg-PEEAs

Synthesis of monomers. The details of the synthesis and characterization of all the prepared monomers and polymers of Arg-PEEAs were given here. Three types of di-p-nitrophenyl esters of dicarboxylic acids (monomer (I), NSu, NA and NS) were synthesized here and the details of the synthesis and characterization have been reported previously.

The p-toluenesulfonic acid salt of L-arginine diester (II) from oligoethylene glycols are newly developed for the first time. Twelve types of new monomers II were prepared and the differences among these monomers II are the salt type (toluenesulfonic acid salt (S type) and hydrochloride salt (Cl type)) and ethylene glycol unit length (y) in the diol part between the two adjacent ester groups: number of ethylene glycol units varies from 2 to 12. The chemical structures of these 12 types of Arg-based monomers II were all confirmed by ¹H NMR, FTIR and solubility tests. All the synthesized bis(L-arginine) diesters are very moisture sensitive and should be stored under vacuum at room temperature.

At room temperature, solubility tests showed that these bis(L-arginine) diesters have very good solubility in polar solvents, such as water, DMSO, DMF; but insoluble in nonpolar or weakly polar solvents, such as isopropanol, acetone, and ethyl acetate. However, it was found that isopropanol can dissolve the monomers II at 50° C. or higher temperature.

The following are some ¹H NMR and FTIR details for the monomers with S salt type. The ¹H NMR data for Cl salt type monomer is same as the corresponding S salt type monomer except for the difference of integration area for some groups:
Arg-2E-S: Yield of purified product: 81%. Appearance: amorphous white powder. IR (cm⁻¹): 1735 [—C(O)—], 1177 [—O—], 1127 [—CH₂—O—CH₂—]; ¹HNMR (DMSO-d₆, ppm, δ): 1.61 [4H, —CH₂—CH₂—CH₂—NH—], 1.77 [4H, —OC(O)—CH(NH₃⁺)CH₂—(CH₂)₂—], 2.29 [6H, H₃C—Ph—SO₃—], 3.10 [4H, —(CH₂)₂—CH₂—NH—], 3.60 [4H, —(O)C—O—CH₂—CH₂—O—], 4.07 [2H, ⁺H₃N—CH(R)—C(O)—O—], 4.32 [4H, —(O)C—O—CH₂—], 7.13, 7.48 [16H, Ph], 7.59 [10H, —CH₂—NH(NH₂⁺)—NH₂], 8.42 [6H, ⁺H₃N—CH(R)—C(O)—O—];
Arg-3E-S: Yield of purified product: 85%. Appearance: amorphous white powder. IR (cm⁻¹): 1736 [—C(O)—], 1178 [—O—], 1125 [—CH₂—O—CH₂—]; ¹HNMR (DMSO-d₆, ppm, δ): 1.63 [4H, —CH₂—CH₂—CH₂—NH—], 1.78 [4H, —OC(O)—CH(NH₃⁺)CH₂—(CH₂)₂—], 2.28 [6H, H₃C—Ph—SO₃—], 3.12 [4H, —(CH₂)₂—CH₂—NH—], 3.55-65 [8H, —(O)C—O—CH₂—CH₂—O—CH₂—], 4.09 [2H, ⁺H₃N—CH(R)—C(O)—O—], 4.31 [4H, —(O)C—O—CH₂—], 7.15, 7.49 [16H, Ph], 7.62 [10H, —CH₂—NH(NH₂⁺)—NH₂], 8.47 [6H, ⁺H₃N—CH(R)—C(O)—O—];
Arg-4E-S: Yield of purified product: 87%. Appearance: amorphous white powder. IR (cm⁻¹): 1734 [—C(O)—], 1179 [—O—], 1124 [—CH₂—O—CH₂—]; ¹H NMR (DMSO-d₆, ppm, δ): 1.62 [4H, —CH₂—CH₂—CH₂—NH—], 1.79 [4H, —OC(O)—CH(NH₃⁺)CH₂—(CH₂)₂—], 2.27 [6H, H₃C—Ph—SO₃—], 3.11 [4H, —(CH₂)₂—CH₂—NH—], 3.60-70 [12H, —(O)C—O—CH₂—CH₂—O—CH₂—CH₂—], 4.08 [2H, ⁺H₃N—CH(R)—C(O)—O—], 4.30 [4H, —(O)C—O—CH₂—], 7.17, 7.50 [16H, Ph], 7.63 [10H, —CH₂—NH(NH₂⁺)—NH₂], 8.49 [6H, ⁺H₃N—CH(R)—C(O)—O—];
Arg-6E-S: Yield of purified product: 89%. Appearance: amorphous white viscous solid. IR (cm⁻¹): 1737 [—C(O)—], 1177 [—O—], 1127 [—CH₂—O—CH₂—]; ¹H NMR (DMSO-d₆, ppm, δ): 1.63 [4H, —CH₂—CH₂—CH₂—NH—], 1.80 [4H, —OC(O)—CH(NH₃⁺)CH₂—(CH₂)₂—], 2.29 [6H, H₃C—Ph—SO₃—], 3.14 [4H, —(CH₂)₂—CH₂—NH—], 3.60-70 [20H, —(O)C—O—CH₂—CH₂—O—(CH₂—CH₂—)₂], 4.10 [2H, ⁺H₃N—CH(R)—C(O)—O—], 4.32 [4H, —(O)C—O—CH₂—], 7.16, 7.50 [16H, Ph], 7.64 [10H, —CH₂—NH(NH₂⁺)—NH₂], 8.50 [6H, ⁺H₃N—CH(R)—C(O)—O—]; and
Arg-12E-S: Yield of purified product: 84%. Appearance: amorphous white viscous solid. IR (cm⁻¹): 1737 [—C(O)—], 1177 [—O—], 1124 [—CH₂—O—CH₂—]; ¹H NMR (DMSO-d₆, ppm, δ): 1.61 [4H, —CH₂—CH₂—CH₂—NH—], 1.78 [4H, —OC(O)—CH(NH₃⁺)CH₂—(CH₂)₂—], 2.29 [6H, H₃C—Ph—SO₃—], 3.10 [4H, —(CH₂)₂—CH₂—NH—], 3.60-70 [44H, —(O)C—O—CH₂—CH₂—O—(CH₂—CH₂—)₅], 4.09 [2H, ⁺H₃N—CH(R)—C(O)—O—], 4.31 [4H, —(O)C—O—CH₂—], 7.15, 7.49 [16H, Ph], 7.61 [10H, —CH₂—NH(NH₂⁺)—NH₂], 8.47 [6H, ⁺H₃N—CH(R)—C(O)—O—].

Synthesis of Arg-PEEA Polymers. The Arg-PEEAs (FIG. 2) were synthesized in the p-toluenesulfonic acid salt or chlorine salt form, while all other PEAs from prior reported studies were not in any salt form. This is because of the strong base nature of the guanidine group in L-Arginine. The guanidine group has a much higher pKa than the amine groups of PEI and PLL-HBr, suggesting a stronger interaction with anionic DNA chain. The p-toluenesulfonic acid counter ion or hydrochloride acid counter ion, however, were found not to adversely affect the DNA binding capability of Arg-PEEAs, and the following cytotoxicity tests showed that all the Arg-PEEAs are nontoxic to the cells even at large dosages.

The reaction conditions for Arg-PEEA synthesis were determined in terms of reaction temperature and time, catalyst and its concentration, the molar ratio between 2 monomers, monomer concentration. After testing, we found that desirable polycondensation reaction conditions for the Arg-PEEAs are: reaction temperature: 75° C.; duration: 48 hours, concentration of each monomer: 1.0-1.5 mol/L; the reaction medium: DMSO; catalyst (acid acceptor): NEt₃. The molar ratio of the two monomers (I and II) should be exactly equal to 1.0:1.0, and the molar ratio between the monomer and acid receptor is suggested to be 1.0: 1.1. The final product yields are high (>80%) under the optimized reaction conditions.

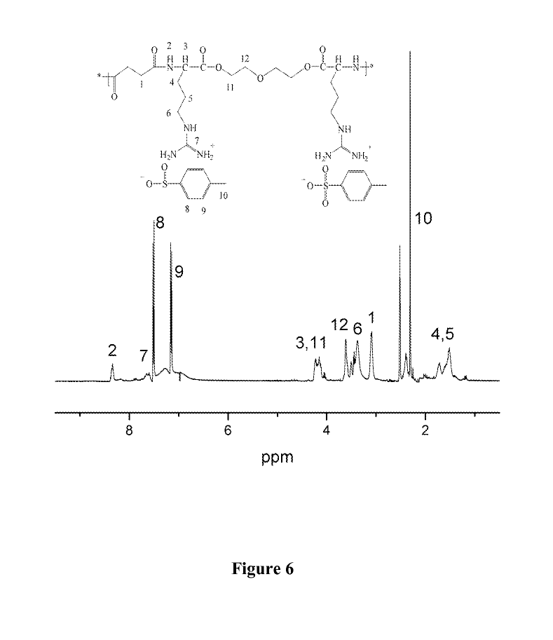

For the chemical structure identification of all the synthesized Arg-PEEAs, their structures were confirmed by both ¹H NMR and FTIR spectra. For FTIR data, the carbonyl bands at 1648-1650 cm⁻¹ (amide I), 1538-1542 cm⁻¹ (amide II), and 1738-1742 cm⁻¹ (ester), and NH vibrations at 3290 cm⁻¹ are typical for all Arg-PEEAs obtained. FIG. 4 showed an example of the ¹H NMR spectrum of 2-Arg-2E-S. All the ¹H NMR peaks of 2-Arg-2E-S were well identified, and the integration area ratio is consistent with the calculated theoretical ratio. The ¹H NMR peaks marked with numbers from 1 to 12 are assigned to the corresponding protons of 2-Arg-2E-S as shown in FIG. 6.

For the thermal property of the Arg-PEEAs, DSC results indicated that the Arg-PEEAs did not have melting points (Tₘ). The glass transition temperature (T_{g}) of Arg-PEEAs were measured. An examination for the effect of the number of methylene groups in the diacid (x) part of the Arg-PEEAs revealed that an increase x led to a decreased T_{g} when y was fixed. For example, if y value was fixed at 2, the T_{g} decreased from 31° C. to 29° C., then to 25° C. when x decreased from 2 to 4, then 6 (2-Arg-2E-S to 4-Arg-2E-S, then to 8-Arg-2E-S). When the y value was increased from 2 to 12 at fixed x value, the same decreasing trend was observed.

According to our data, unsaturated Arg-PEAs (double bonds in the main chain, not the side chain) significantly increases the T_{g} value because of the stiff polymer backbone. For example, the Tg values for 2-Arg-4-S and 2-Arg-2E-S are 46° C. and 31° C., respectively, while 2-Arg-2E-S only has an extra oxygen atom for each repeating unit compared with 2-Arg-4-S. So the introduction of soft oligoethylene glycol chain significantly decreased the Tg value of Arg-PEEAs. Thus, the introduction of soft segment, oligoethylene glycol, to the polymer backbone makes the whole polymer chain structure much softer and more flexible. The T_{g} value decreases if we compare the oligoethylene glycol based Arg-PEER with fatty diol based Arg-PEA.

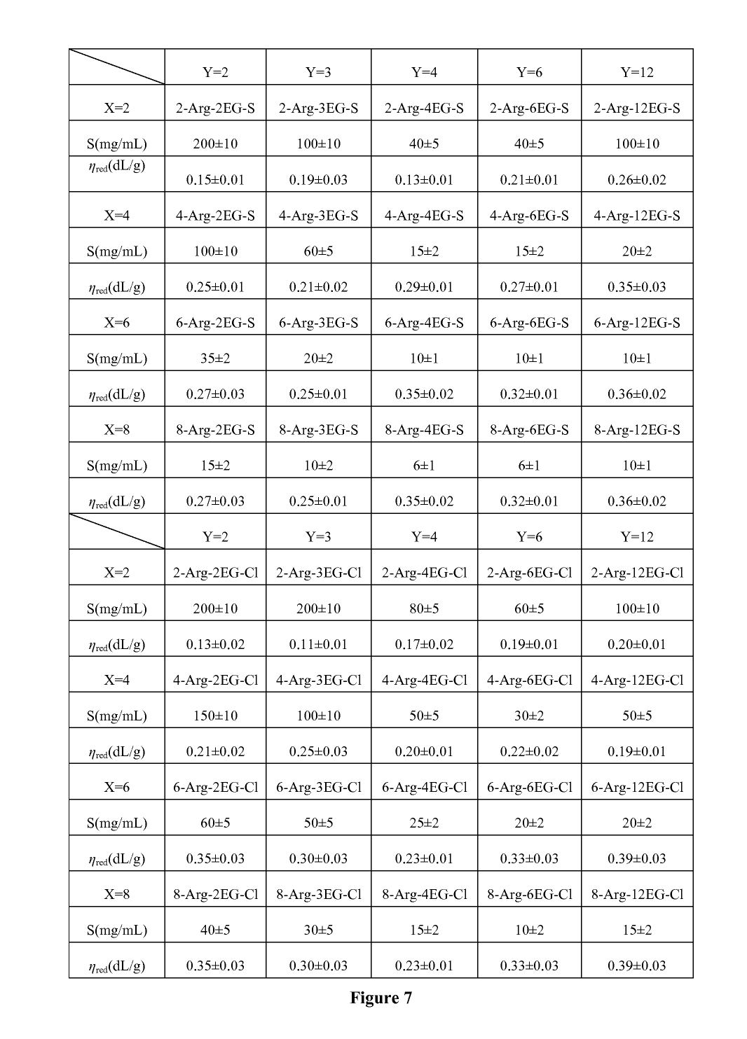

The solubility of Arg-PEEAs in water and common organic solvents at room temperature was tested. Solubility was assessed at 2.0 mg/mL at a room temperature as an index whether a polymer is soluble or not. Due to their strong polar nature, Arg-PEEAs tended to dissolve in polar solvents. All of the Arg-PEEAs synthesized were soluble in polar organic solvents like DMSO, DMF, methanol or water, but did not dissolve in non-polar or weak polar organic solvents like ethyl acetate, THF or chloroform. Quantitative water solubility data for Arg-PEEAs were measured at room temperature (Table 3; FIG. 7). The effect of x and y parameters on Arg-PEEA water solubility revealed that both x and y had a major impact on the water solubility of Arg-PEEAs; and an increase in the methylene chain length in the dicarboxylic acid part (x) reduced the water solubility significantly due to the increasing hydrophobicity. For example, the solubility of Arg-PEEAs decreased from 200 mg/mL to 15 mg/mL as x increased from 2 (2-Arg-2E-S) to 8 (8-Arg-2E-S) at a constant y=2.

A similar solubility—structure relationship was also found with an increase in y (from 2 to 6) at a constant x. It did not work for large y value, such as y=12. So the water solubility of Arg-PEEAs could be used as an index of polymer hydrophilicity/hydrophobicity. By adjusting the x or y, the Arg-PEEA polymers' hydrophilicity/hydrophobicity can be tuned to meet specific needs.

Compared with the saturated aliphatic diol-based Arg-PEAs, Arg-PEEAs showed significant solubility increasing in distilled water due to the introduction of relative hydrophilic and soft ethylene glycol units. For example, the 2-Arg-2E-S showed much higher water solubility than 2-Arg-4-S and the solubility difference is more than 150 mg/mL. And the chemical structure difference of the repeating unit of 2 polymers is only one oxygen atom.

All the prepared Arg-PEEAs were obtained in fairly good yields (>75%) with η_{red} ranging from 0.11 to 0.39 dL/g (Table 3; FIG. 7). Larger η_{red} value means higher molecular weight. The molecular weight data was not available here because all arginine based PEEAs cannot be dissolved in THF, which is the solvent for the central GPC facility available to us.

### Gel Retardation Assay

Gel Retardation Assay is a widely used method for measuring DNA condensing capability of polymeric transfection candidates. The main goal is to determine the proper WR of Arg-PEEA to DNA required for a completely condensing of DNA during the polyplex formation, the first key step toward non-viral gene transfection.

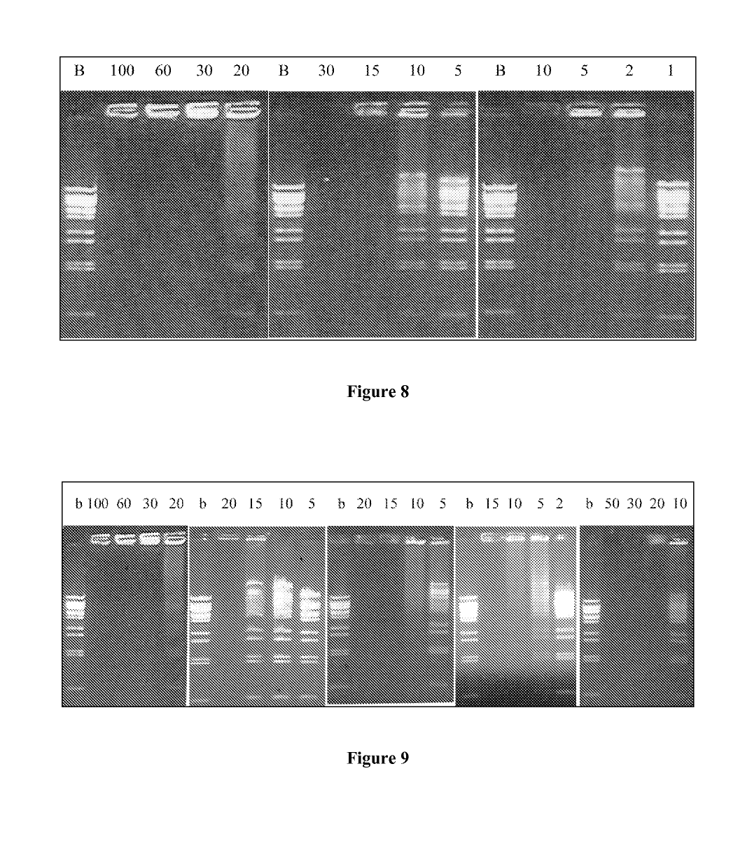

FIGS. 8 and 9 showed some examples of the electrophoresis data for the Arg-PEEA/DNA complexes. These results demonstrated the DNA condensation capability of Arg-PEEAs, and provided the basic formulation information for subsequent transfection experiments. Most important of all, the electrophoresis data showed that different types of Arg-PEEAs (in terms of x and y parameters) required different amounts of Arg-PEEAs for a complete DNA condensation as indicated by the different WR.

In order to have a quantitative comparison of the DNA condensation capability of Arg-PEEAs, the minimum WR of Arg-PEEA to DNA that could completely condense DNA was selected and compared. For example (FIG. 8), 2-Arg-2E-Cl needed a minimum WR of 30 to completely condense the DNA marker; while 4-Arg-2E-Cl and 8-Arg-2E-Cl needed a minimum WR of 15 and 10 for a complete condensation, respectively. So the minimum WR decreased when we increased the x value. The same trend was observed when y value was increased from 2 to 4. This relationship, however, did not hold at a large y values (such as y=6 and 12 (FIG. 9).

We also found that the Arg-PEEA buffer solutions, if stored at 4° C., could retain their DNA condensing capability for around 1 month or even longer time, suggesting there was no obvious structure change or degradation of Arg-PEEAs in the buffer solution at 4° C. It is very important to recognize that a complete Arg-PEEAs dissolution, precise polymer concentration and volume are critical for reproducible data. The Arg-PEEA polymers must be dissolved completely and the volume should be in the range of 2-5 μL to avoid any possible experimental errors. Some Arg-PEEAs have low water solubility and would take an extended time for a complete dissolution.

### Transfection Efficiency

In this example, the plasmid DNA that encodes for a firefly luciferase driven by a collagen promoter was used. By measuring luciferase activities in cell lysates, which in this case is mainly determined by the amount of DNA transferred into the cells, we compared the transfection efficiency of Arg-PEEAs with a commercial transfection agent, Lipofectamine2000®, for determining the transfection feasibility of Arg-PEEAs.

In any transfection protocol development, cell density, transfection time, transfection temperature, transfection media and buffer types are important parameters for optimization to achieve the best transfection data. In the Arg-PEEA/DNA system, the optimized transfection protocol of Arg-PEEA/DNA system was: transfection time: 3-4 hours for cell lines and 12-16 hours for primary cells and stem cells; transfection temperature: 37° C.; transfection media: serum free DMEM media without antibiotics; buffer for Arg-PEEA/DNA: HEPES (20 mM) or PBS buffer (1×); cell density: 10,000-30,000 per well for 24-well cell culture plate. At this optimized condition, it was observed that the luciferase activity could reach the peak value over a range of WR of Arg-PEEA to DNA.

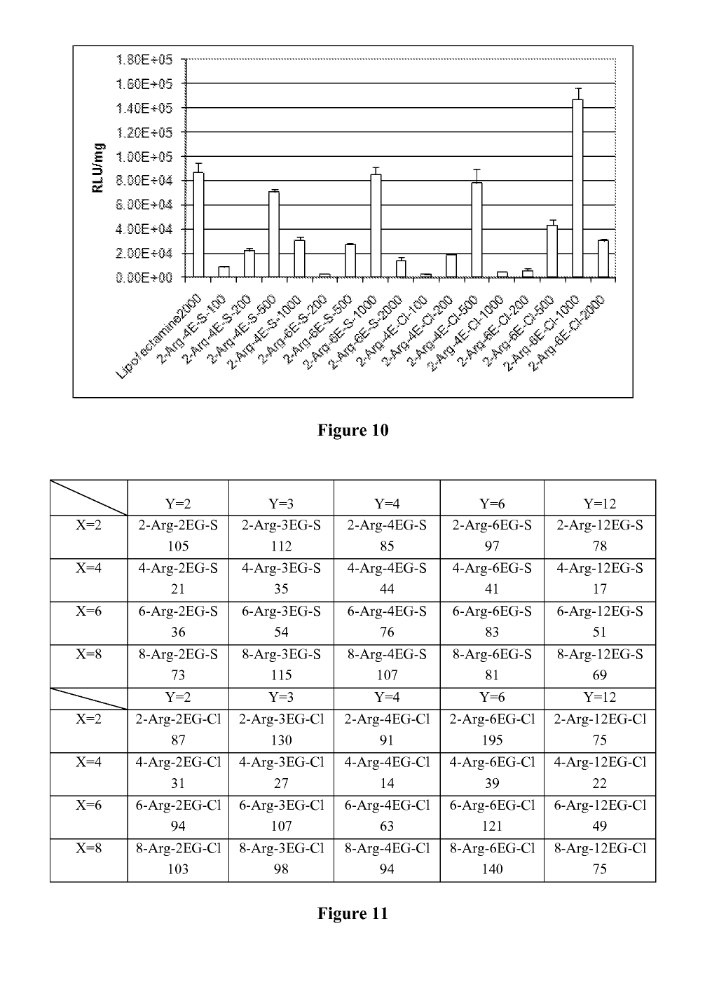

FIG. 10 showed an example of the transfection results from 4 types of Arg-PEEA/DNA at various WR: 2-Arg-4E-S, 2-Arg-6E-S, 2-Arg-4E-Cl and 2-Arg-6E-Cl. The data show that all the Arg-PEEA/DNA complexes show transfection ability over a very broad WR range, and each type of Arg-PEEA/DNA complex showed a peak transfection at a specific WR. For example, the 2-Arg-6E-Cl/DNA showed transfection capability over WR from 200 to 2000, but the highest transfection capability was around the WR of 1000. For the 2-Arg-4E-S/DNA system, the peak transfection, however, occurred at WR 500.

These transfection results also showed that the WR of Arg-PEEAs/DNA to reach the desired transfection efficiency was much higher than the minimal WR required for completely condensing DNA in the electrophoresis data. For example, 2-Arg-6E-Cl need a WR of 20 for completely condensing DNA and need a WR of 1000 for efficient DNA transfection. The transfection agent's DNA condensation capability is known to have a desirable effect on the subsequent gene delivery efficiency, but is not the only factor that is responsible for the outcome of gene delivery efficiency. This may be attributed to the need of excess amounts of Arg-PEEAs required to achieve not only a stable Arg-PEEA/DNA complex system in the transfection media but also provided additional cationic charge to the Arg-PEEA/DNA complex for its proper penetration into the cells membranes. The larger dosage of Arg-PEEA required for the desired transfection, however, didn't impose any adverse cytotoxicity as described later.

To compare the transfection efficiency of all the Arg-PEEAs, the highest or peak RLU/mg (relative light unit/mg) of each polymer was selected and normalized against the RLU/mg value of the commercial control (Lipofectamine2000®), i.e., setting the RLU/mg value of the control at 100 (Table 4; FIG. 11). This normalization process removed the batch to batch variation. The normalized transfection data in Table 4 (FIG. 11) showed that many of these Arg-PEEAs had comparable or better transfection efficiency (i.e., those Arg-PEEAs having 100 or greater normalized values) than the commercial transfection reagent Lipofectamine®. Those Arg-PEEAs having lower x and medium y values were the most favorable for higher transfection efficiency, while high y value, especially y=12, is not favorable for high transfection.

### GFP Expression

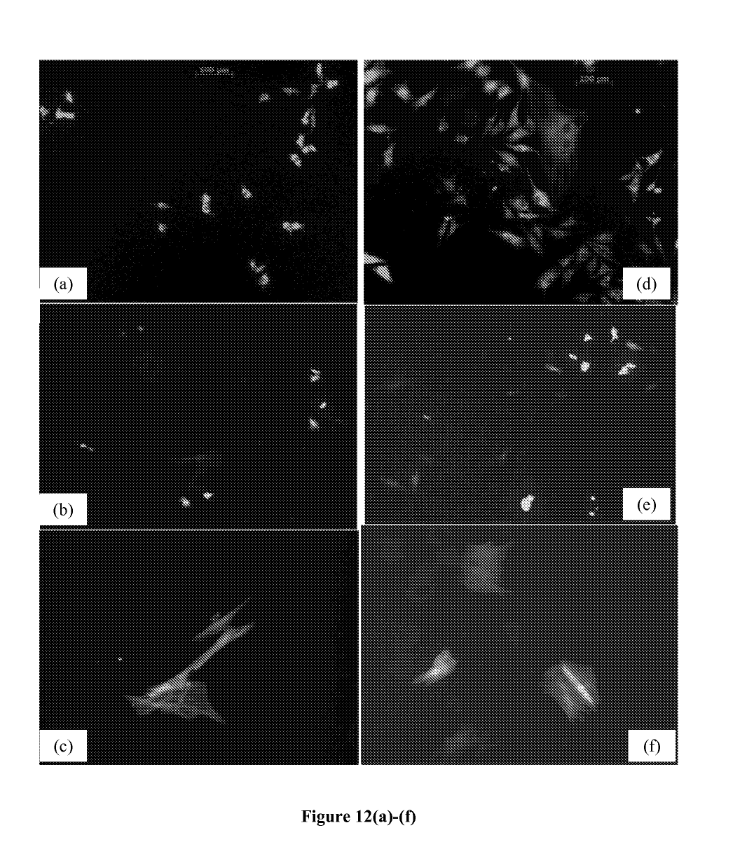

To visually confirm the transfection efficiency obtained from the luciferase activity data, all the cells were transfected by plasmid DNAs encoding for green fluorescent protein (GFP). Two days following the transfection, the cells were examined under a fluorescence microscope for their GFP expression (transfected cells show green). FIG. 12 shows GFP plasmid DNAs were successfully expressed inside different cell types as commercial transfection agent Lipofectamine2000® did.

### Zeta Potential and Particle Size Measurements for Arg-PEEA/DNA Complex

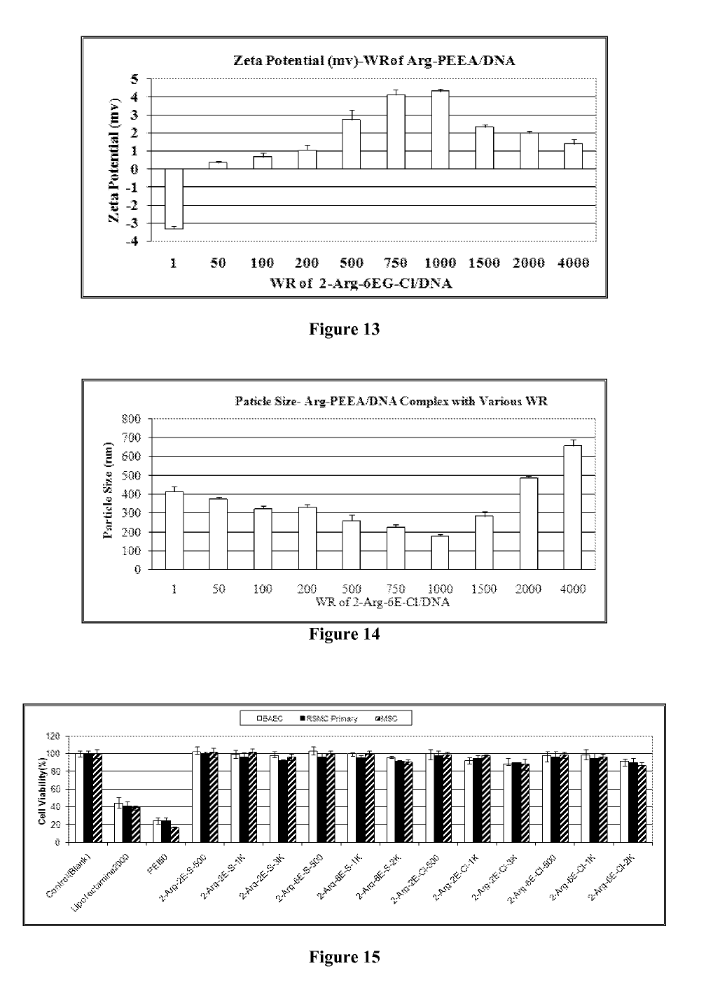

The Zeta potential measurement was used to study the charge property and the charge-structure relationship of the Arg-PEEA/DNA complex. FIG. 13 showed the zeta potentials of some Arg-PEEA/DNA complexes as a function of the weight ratio of Arg-PEEA to DNA.

For example, the data in FIG. 13 (2-Arg-6E-Cl) could be divided into 3 regions, depending on the ratio of Arg-PEEA to DNA. As the weight ratio of Arg-PEEA to DNA increased, the zeta potential of the complex increased (from negative to positive), suggesting that as more Arg-PEEAs added into the DNA, the charge property of the complex changed from negative to positive. A further increase in the weight ratio of Arg-PEEA to DNA, the zeta potential of the complex reached a peak, (WR is around 1000), and a further increase in the WR resulted in a reduction in zeta potential of the complex. The WR with a peak zeta potential suggests that the Arg-PEEA/DNA complex must be in the most stable state, and should be the desired condition for gene transfection, which is consistent with transfection data.

The particle size measurement was used to study the particle size of Arg-PEEA/DNA complex in the buffer solution and the size-structure relationship of the Arg-PEEA/DNA complex. FIG. 14 showed the particle sizes of some Arg-PEEA/DNA complexes as a function of the weight ratio of Arg-PEEA to DNA.

For example, the data in FIG. 14 (2-Arg-6E-Cl) could be divided into 3 regions, depending on the ratio of Arg-PEEA to DNA. As the weight ratio of Arg-PEEA to DNA increased, the particle size of the complex decreased, suggesting that as more Arg-PEEAs added into the DNA, the DNA molecules were going to collapse. A further increase in the weight ratio of Arg-PEEA to DNA, the particle size of the complex reached bottom value, (WR is around 1000), and a further increase in the WR resulted in a increase in particle size of the complex. The WR with a bottom particle size suggests that the Arg-PEEA/DNA complex must be in the most stable state, and should be the desirable condition for gene transfection, which is consistent with transfection data.

### Cytotoxicity of Arg-PEEA/DNA Complex by MTT Assay

Cytotoxicity of Arg-PEEA/DNA complexes was evaluated by MTT assay. The MTT system is a simple, accurate, reproducible means of detecting living cells via mitochondrial dehydrogenase activity. An increase in cell number (cell proliferation) results in an increase in the amount of MTT formazan formed and an increase in UV absorbance. PEI, Lipofectamine2000® were used as the controls. All the synthesized Arg-PEEAs at different WR of Arg-PEEA/DNA were tested by MTT assay and some of the results were shown in FIG. 15. Three types of cells were used for MTT assay and they are BAEC, RSMC Primary and MSC. The MTT data clearly demonstrated that at 12 hour treatment, all the Arg-PEEA/DNA complexes showed very little toxicity to the tested cells even at a very large dosage. Although the 2 controls (Lipofectamine2000® and PEI) required lower dosages than Arg-PEEAs to reach optimum transfection efficiency, they still showed a significantly higher cytotoxicity than Arg-PEEAs. Since Arg-PEEA had a lower positive charge density than the 2 control transfection reagents, a larger dose of Arg-PEEA was needed to achieve efficient transfection. The statistical data analysis showed that there is no significant difference of any Arg-PEEA treatment compared to the control at the p value of 0.05 level by Dunnet test of planned comparison. So there is no evidence of toxicity of Arg-PEEAs.

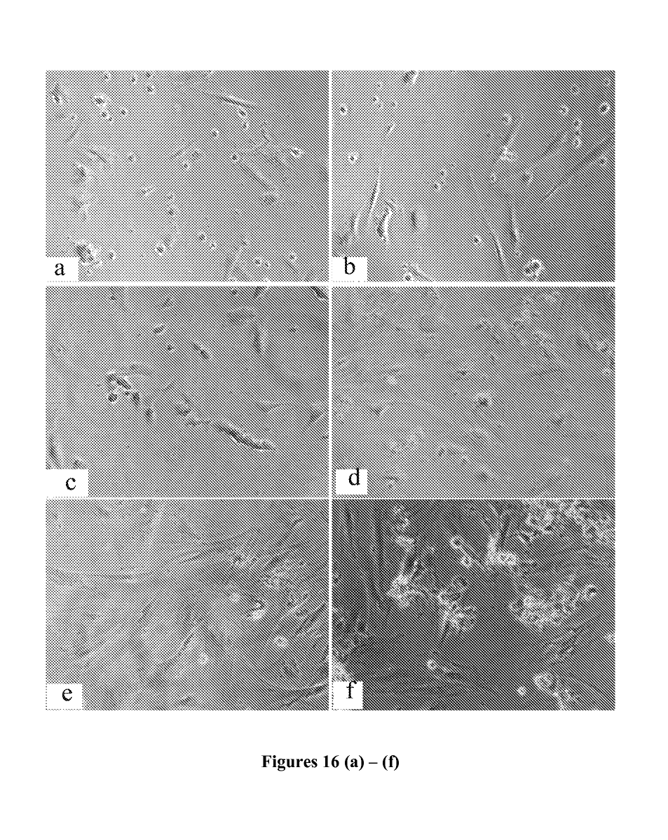

The cytotoxicity of Arg-PEEA/DNA complex can also be confirmed by observing cell morphology under light microscope as shown in FIG. 16 in addition to MTT assay. FIG. 16 showed the images of HUVEC primary cells and MSC stem cells 48 h after treatment of different Arg-PEEA/DNA complexes for 12 hours. It can be seen that the cells treated by Arg-PEEA/DNA displayed normal HUVEC and MSC morphology, confirming the nontoxic nature of these Arg-PEEAs. In contrast, those HUVEC primary cells and SMC stem cells transfected with Lipofectamine2000® appeared to be somewhat unhealthy. So we can conclude that these newly developed Arg-PEEAs are non-toxic and very safe to a variety of different cell types.

### Conclusion

We prepared a series of water soluble, biocompatible and biodegradable L-Arginine and oligoethylene glycol based poly (ester amide)s (Arg-PEEAs), then studied their feasibility as a gene delivery system for a variety of cell types, from cell lines to primary cells and stem cells. The relationship of polymer structure-function was investigated in terms of the number of methylene and ethylene glycol units. Through various assays and methods, we confirmed that Arg-PEEAs could condense the DNA and form stable complexes. Certain Arg-PEEAs showed better transfection efficiency than Lipofectamine2000®, while with a much lower cytotoxicity. The polymer structure-function quantitative relationship was revealed to certain degree. This new Arg-PEEA family showed great potential as a better and safer transfection agent.

### Example 2

### Synthesis, Characterization, and Use of Poly(ester amide)s as Transfection Agents

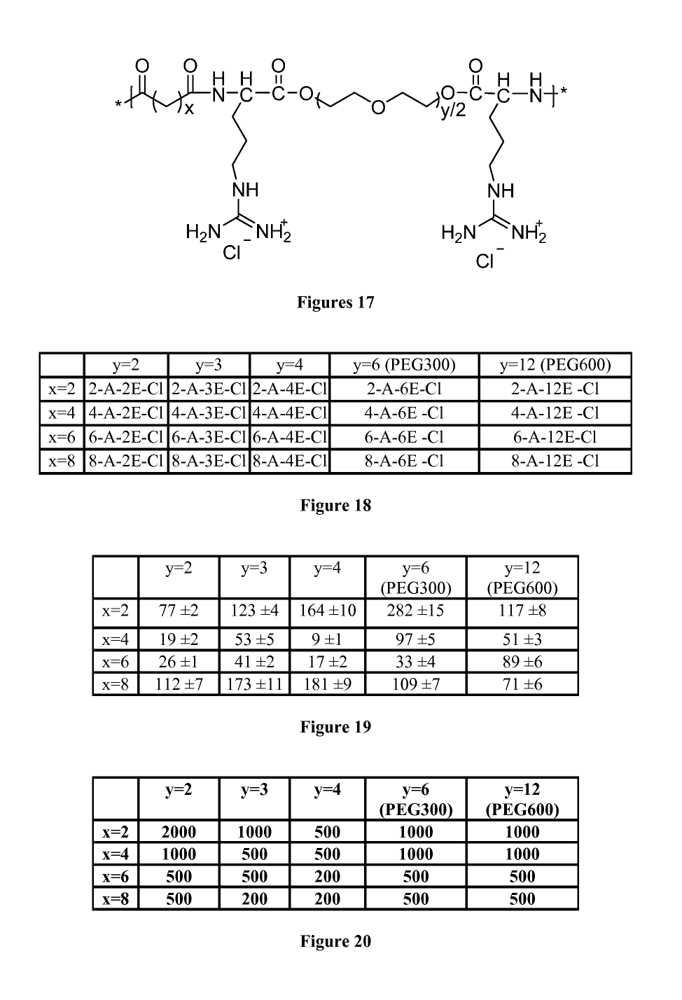

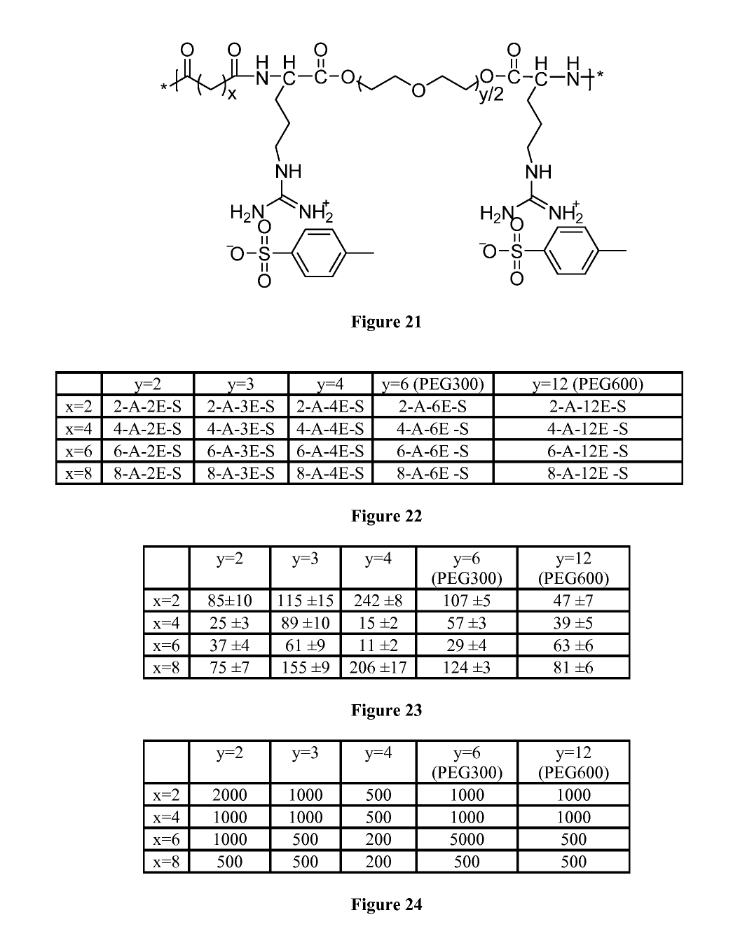

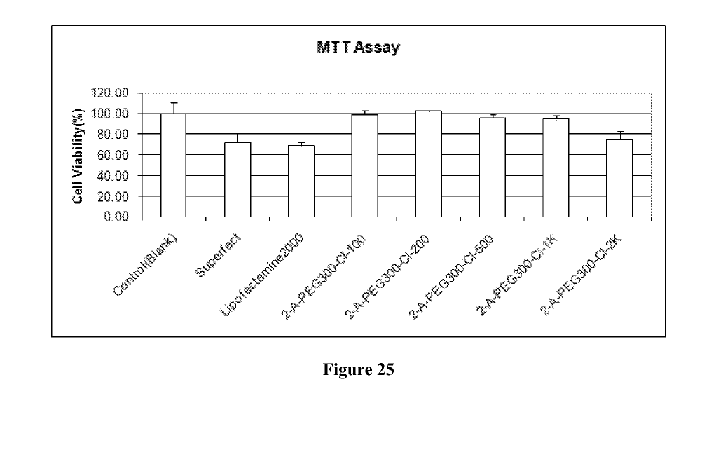

The PEEAs described in this example were synthesized, characterized and used as described in Example 1. FIGS. 17-25 describe examples of PEEAs and use thereof as transfection agents.

While the invention has been particularly shown and described with reference to specific embodiments (some of which are preferred embodiments), it should be understood by those having skill in the art that various changes in form and detail may be made therein without departing from the spirit and scope of the present invention as disclosed herein.

What is claimed is:

1) A polymer having the following structure: 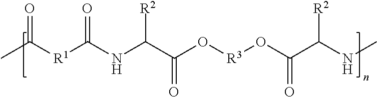
wherein n is an integer from 2 to 100;
wherein R¹ at each occurrence in the polymer is independently selected from C₂ to C₂₀ alkyl or alkenyl;
wherein R² at each occurrence in the polymer is independently selected from —(CH₂)_{q}NHC(═NH₂⁺)NH₂, wherein q at each occurrence in the polymer is from 1 to 5, 4-alkylene imidazolium and —(CH₂)ₜNH₃⁺, wherein t at each occurrence in the polymer is from 1 to 5, and
wherein R³ is an alkyl polyether, wherein the alkyl moiety of the alkyl polyether at each occurrence in polymer comprises from 1 to 8 carbons.

2) The polymer of claim 1, wherein the polymer has the following structure: 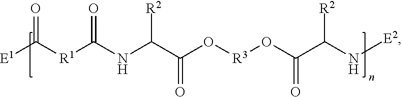
wherein E¹ and E² are independently selected from H and OH.

3) The polymer of claim 1, wherein the alkyl polyether is —(CH₂—CH₂—O)ₘ—CH₂—CH₂— and m at each occurrence in the polymer is an integer from 1 to 100.

4) The polymer of claim 1, wherein the polymer is present as a halide, Tos⁻, acetate, halogen-substituted acteate, sulfate, nitrate, or a combination thereof salt.

5) The polymer of claim 1, wherein the polymer has a molecular weight of from 2000 g/mol to 100,000 g/mol.

6) A composition comprising the polymer of claim 1.

7) The composition of claim 6, further comprising biological molecule selected from a poly nucleic acid, negatively-charged protein, negatively-charged polysaccharide and a combination thereof.

8) The composition of claim 7, wherein the poly nucleic acid comprises a gene encoding a peptide or polypeptide.

9) The composition of claim 7, wherein the poly nucleic acid is RNA.

10) The composition of claim 7, wherein the RNA is an antisense poly nucleic acid.

11) The composition of claim 7, wherein the weight ratio of polymer to poly nucleic acid is from 50:1 to 12,000:1.

12) The composition of claim 7, wherein the polymer and poly nucleic acid form a complex and form particles having a size of from 50 nm to 1000 nm.

13) The composition of claim 12, wherein the particles have a size of from 150 nm to 250 nm.

14) A method for intracellular delivery of a biomolecule comprising:
a) contacting a cell with the composition of claim 7 under conditions suitable to deliver a biomolecule into a cell.

15) The method of claim 15, wherein the intracellular delivery of a biomolecule is transfection a poly nucleic acid into a cell.

16) The method of claim 14, wherein the composition further comprises a biomolecule selected from a poly nucleic acid, negatively-charged protein, negatively-charged polysaccharide and a combination thereof.

17) The method of claim 16, wherein the poly nucleic acid comprises a gene encoding a peptide or polypeptide.

18) The method of claim 16, wherein the poly nucleic acid is RNA.

19) The method of claim 16, wherein the RNA is an antisense poly nucleic acid.

20) The method of claim 14, wherein the cell is a primary cell or stem cell.
